(19) United States
(12) Patent Application Publication
(10) Pub. No.: US 2023/0391809 A1
(43) Pub. Date: Dec. 7, 2023

(54) HYDROPHOBIC COMPOSITIONS INCLUDING A HYDROPHILIC ACTIVE INGREDIENT AND METHODS RELATING THERETO

(71) Applicant: BioMimetix JV, LLC, Englewood, CO (US)

(72) Inventors: James D. Crapo, Englewood, CO (US); Kimberly C. Stone, Greenwood Village, CO (US); Keith Arthur Johnson, Durham, NC (US)

(21) Appl. No.: 18/330,405

(22) Filed: Jun. 7, 2023

Related U.S. Application Data
(60) Provisional application No. 63/349,793, filed on Jun. 7, 2022.

(51) Int. Cl. C07F 13/00; C07D 487/22; A61K 45/06; A61K 9/00; A61K 9/06; A61K 47/06; A61K 47/44

(52) U.S. Cl. CPC C07F 13/005; C07D 487/22; A61K 45/06; A61K 9/0014; A61K 9/06; A61K 47/06; A61K 47/44

(57) ABSTRACT

Described herein are hydrophobic compositions including a hydrophilic active ingredient and to methods of making and using the same.

### FIELD

The present invention generally relates to hydrophobic compositions including a hydrophilic active ingredient and to methods of making and using the same.

### BACKGROUND

Topical drug delivery can provide localized treatment and/or delivery of an active ingredient and can limit systemic exposure and/or adverse effects. However, not all active ingredients have physicochemical properties suitable for topical delivery. For example, typically it is desirable that an active ingredient be soluble in a topical formulation, have a chemical stability that allows the formulation to be stored at room temperature, and for the active agent to cross the stratum corneum and provide a local therapeutic response upon topical administration of the formulation. Generally, active agents that are ideal for topical delivery have: (1) a molecular weight of 500 Daltons or less, (2) a calculated log P (c log P) value in a range of 1.0-4.0, (3) a topological polar surface area of 100 Å² or less, and (4) two or less aromatic rings, and active agents whose properties fall outside two or more of these four ranges are highly likely to have topical formulation challenges. Accordingly, hydrophilic active agents, such as those with a c log P of less than 1.0, can be challenging to formulate for topical delivery. For example, BMX-010, a highly hydrophilic meso-substituted metalloporphyrin, has a molecular weight of 965 Daltons and a c log P of −6.9, and has been challenging to formulate for topical administration with sufficient delivery to the epidermis and dermis. In addition, formulating BMX-010 and other meso-substituted metalloporphyrins has aesthetic challenges, particularly in regard to the color of the formulation as cream or gel formulations of BMX-010 can provide a yellow color on application to the skin and/or can have a purple color prior to application.

### SUMMARY

One aspect of the present invention is directed to a composition comprising: a hydrophilic active ingredient (e.g., a hydrophilic active ingredient having a log P of less than 1), optionally wherein the hydrophilic active ingredient is a redox active compound that comprises a metal, further optionally wherein the hydrophilic active ingredient is present in the composition in an amount of about 0.005%, 0.01%, 0.05%, 0.1%, or 0.5% to about 1%, 2%, 5%, or 10% by weight of the composition; a hydrophobic carrier (e.g., petrolatum), optionally wherein the hydrophobic carrier is present in an amount of about 60% to about 99% by weight of the composition; and an acid, optionally wherein the acid is present in the composition in an amount of about 0.001%, 0.005%, 0.01%, 0.05%, or 0.1% to about 0.2%, 1%, 2%, or 2.5% by weight of the composition.

A further aspect of the present invention is directed to a composition comprising: a hydrophilic active ingredient (e.g., a hydrophilic active ingredient having a log P of less than 1), optionally wherein the hydrophilic active ingredient is a redox active compound that comprises a metal, further optionally wherein the hydrophilic active ingredient is present in the composition in an amount of about 0.005%, 0.01%, 0.05%, 0.1%, or 0.5% to about 1%, 2%, 5%, or 10% by weight of the composition; a hydrophobic carrier (e.g., petrolatum), optionally wherein the hydrophobic carrier is present in an amount of about 60% to about 99% by weight of the composition; a stiffening agent (e.g., a wax such as paraffin wax), optionally wherein the stiffening agent is present in the composition in an amount of about 1% to about 10% by weight of the composition; and an acid, optionally wherein the acid is present in the composition in an amount of about 0.001%, 0.005%, 0.01%, 0.05%, or 0.1% to about 0.2%, 1%, 2%, or 2.5% by weight of the composition.

Another aspect of the present invention is directed to a method of preparing a composition of the present invention, the method comprising: combining a hydrophobic carrier, an acid, and a hydrophilic active ingredient (e.g., a redox active compound that comprises a metal), thereby providing the composition. In some embodiments, the method comprises combining a hydrophobic carrier (e.g., a hydrocarbon carrier such as a petrolatum), a stiffening agent (e.g., a wax), an acid, a hydrophilic active ingredient (e.g., a redox active compound that comprises a metal), and optionally an oil, thereby providing the composition.

A further aspect of the present invention is directed to a method of treating and/or preventing a disease, disorder, and/or condition (e.g., a dermatological condition) in a subject, the method comprising topically administering to the skin of the subject a composition of the present invention.

It is noted that aspects described with respect to one embodiment may be incorporated in different embodiments although not specifically described relative thereto.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1 is graph of the median amount of BMX-010 in tissue, ng/cm², versus the amount of BMX-010 in an ointment by weight of the ointment.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

“Alkyl” as used herein alone or as part of another group, refers to a straight or branched chain saturated hydrocarbon containing from 1 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “alkyl” or “lower alkyl” is intended to include both substituted and unsubstituted alkyl or lower alkyl unless otherwise indicated and these groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Alkenyl” as used herein alone or as part of another group, refers to a straight or branched chain hydrocarbon containing from 1 to 20 carbon atoms (or in lower alkenyl 1 to 4 carbon atoms) which include one or more (e.g., 1 to 19) double bonds in the chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “lower alkenyl” is intended to include both substituted and unsubstituted alkenyl or lower alkenyl unless otherwise indicated and these groups may be substituted with groups as described in connection with alkyl and lower alkyl above.

“Alkynyl” as used herein alone or as part of another group, refers to a straight or branched chain hydrocarbon containing from 1 to 20 carbon atoms (or in lower alkynyl 1 to 4 carbon atoms) which include at least 1 triple bond in the chain. Representative examples of alkynyl include, but are not limited to, 2-propynyl, 3-butynyl, 2-butynyl, 4-pentynyl, 3-pentynyl, and the like. The term “alkynyl” or “lower alkynyl” is intended to include both substituted and unsubstituted alkynyl or lower alkynyl unless otherwise indicated and these groups may be substituted with the same groups as set forth in connection with alkyl and lower alkyl above.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or lower alkyl.

“Heterocyclic group” or “heterocyclo” as used herein alone or as part of another group, refers to an aliphatic (e.g., fully or partially saturated heterocyclo) or aromatic (e.g., heteroaryl) monocyclic- or a bicyclic-ring system having as ring members atoms of at least two different elements. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and lower alkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” as used herein is as described in connection with heterocyclo above.

“Alkoxy” as used herein alone or as part of another group, refers to an alkyl or lower alkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Formyl” as used herein refers to a —C(O)H group.

“Carboxylic acid” as used herein refers to a —C(O)OH group.

“Hydroxyl” as used herein refers to an —OH group.

“Nitro” as used herein refers to an —NO₂ group.

“Acyl” as used herein alone or as part of another group refers to a —C(O)R radical, where R is any suitable substituent such as aryl, alkyl, alkenyl, alkynyl, cycloalkyl or other suitable substituent as described herein.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRaRb, where Ra and Rb are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl” as used herein refers to a compound of the formula —S(O)(O)R, where R is any suitable substituent such as alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl.

“Urea” as used herein alone or as part of another group refers to an —N(R_{c})C(O)NRₐR_{b} radical, where Rₐ, R_{b} and R_{c} are any suitable substituent such as H, alkyl, cycloalkyl, alkenyl, alkynyl or aryl.

“Alkoxyacylamino” as used herein alone or as part of another group refers to an —N(Ra)C(O)ORb radical, where Ra, Rb are any suitable substituent such as H, alkyl, cycloalkyl, alkenyl, alkynyl or aryl.

“Aminoacyloxy” as used herein alone or as part of another group refers to an —OC(O)NRaRb radical, where Ra and Rb are any suitable substituent such as H, alkyl, cycloalkyl, alkenyl, alkynyl or aryl.

“Pharmaceutically acceptable” as used herein means that the compound, anion, or composition is suitable for administration to a subject to achieve the treatments described herein, without unduly deleterious side effects in light of the severity of the disease and necessity of the treatment.

Provided according to embodiments of the present invention are compositions comprising a hydrophilic active ingredient. A “hydrophilic active ingredient” as used herein refers to a compound that has a log P (log P) of less than 1 and that can provide a pharmacological activity, a direct effect on the treatment and/or prevention of a disease, disorder, and/or condition in and/or on a subject, and/or that can affect the structure and/or a function of the body of a subject. In some embodiments, log P may be a calculated log P (c log P) and/or a predicted log P (p log P). A log P may be calculated and/or determined using methods known in the art such as, but not limited to those described in Kos et al. Free Radical Biology & Medicine 47 (2009) 72-78 and/or Santos et al. International Journal of Pharmaceutics 604 (2021) 120750, the contents of each of which are incorporated herein by reference in their entirety. In some embodiments, a hydrophilic active ingredient can provide a therapeutic benefit to a subject (e.g., a therapeutically useful response in a subject) such as when administered in a therapeutically effective amount; the therapeutic benefit may not be complete or curative, as long as some benefit is provided to the subject. In some embodiments, a hydrophilic active ingredient has a log P of less than 1 to about −10 such as a log P of about 0.8, 0, −1, −1.5, −2, −2.5, −3, −3.5, −4, −4.5, −5, −5.5, −6, −6.5, −7, −7.5, −8, −8.5, −9, −9.5, or −10. In some embodiments, a hydrophilic active ingredient has a molecular weight of about 100, 200, 300, 400, or 500 Daltons to about 600, 700, 800, 900, 1000, 1100, or 1200 Daltons. In some embodiments, a hydrophilic active ingredient has a molecular weight of about 500 Daltons or less. In some embodiments, a hydrophilic active ingredient has a molecular weight of greater than 500 Daltons, optionally greater than about 600, 700, 800 or 900 Daltons. In some embodiments, a hydrophilic active ingredient has a molecular weight in a range of about 600, 700, 800, or 900 Daltons to about 1000, 1100, or 1200 Daltons. In some embodiments, a hydrophilic active ingredient has 0, 1, 2, 3, or 4 aromatic rings. In some embodiments, a hydrophilic active ingredient is a redox active compound that comprises a metal such as, but not limited to, a meso-substituted metalloporphyrin. One or more (e.g., 1, 2, 3, 4, or more) hydrophilic active ingredient(s) may be present in a composition of the present invention.

In some embodiments, a meso-substituted metalloporphyrin of the present invention is Ames negative. Exemplary meso-substituted metalloporphyrins include, but are not limited to, those having a structure of Formula I:

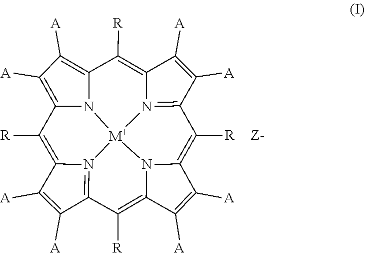

wherein:
each R is, independently, substituted or unsubstituted aryl, heteroaryl, cycloalkyl, or heterocycloalkyl;
each A is, independently, a hydrogen, an electron-withdrawing, or an electron donating group (e.g., is halogen, —NO₂ or —CHO);
M is a metal (e.g., selected from the group consisting of manganese, iron, copper, cobalt, nickel, and zinc) or is absent (in which case a hydrogen is added to two nitrogens as required to correct valency); and
Z⁻ is a counterion (e.g., an anion such as a halogen ion (e.g., chloride, fluoride, bromide, and/or iodide ion) and/or a conjugate base of an acid (e.g., an oleate)).

In some embodiments, each R in the compound of Formula I is a heteroaryl or heterocycloalkyl. In some embodiments, each R in the compound of Formula I is a heteroaryl or heterocycloalkyl that each independently include at least one or two nitrogen atoms in the heterocyclic ring, optionally wherein each R is independently selected from a pyrrolyl, imidazolyl, triazolyl, pyridyl, pyrimidyl, triazinyl, oxazolyl, thiazolyl, oxazinyl, thiazinyl, and/or oxathiazinyl, each of which may be substituted or unsubstituted. In some embodiments, in a compound of Formula I, each R is a heteroaryl or heterocycloalkyl that includes at least one nitrogen atom (or in some embodiments at least two nitrogen atoms) that is substituted (e.g., quaternized) with a substituent such as described in connection with heterocyclic groups above (e.g., substituted with alkyl, alkoxyalkyl, etc.). In some embodiments, Z⁻ in a compound of Formula I is a halogen such as chlorine. In some embodiments, Z⁻ in a compound of Formula I is a conjugate base of an acid such as an oleate.

In some embodiments, M is absent in the compound of Formula I, so the compound is in its free base form and has a structure of Formula I″:

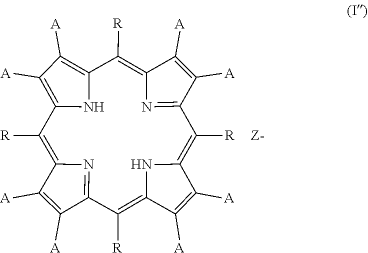

wherein A, R, and Z are each as defined above.

In some embodiments, the meso-substituted metalloporphyrin is an alkyl substituted imidazole porphyrin. Exemplary alkyl substituted imidazole porphyrins include, but are not limited to, those having a structure of Formula A1 or A2:

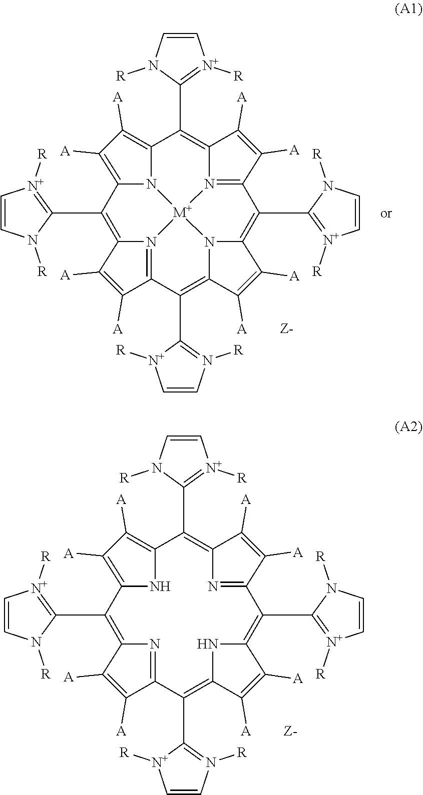

wherein:
each R is, independently, a C₁₋₁₂ alkyl (straight chain or branched), more preferably C₂₋₆alkyl, and most preferably ethyl, propyl, butyl, or pentyl (straight chain or branched);
each A is, independently, a hydrogen or an electron withdrawing group (e.g., halogen, —NO₂ or —CHO);
M is a metal selected from the group consisting of manganese, iron, copper, cobalt, nickel and zinc; and
Z⁻ is a counterion.

In some embodiments, the meso-substituted metalloporphyrin has the structure:

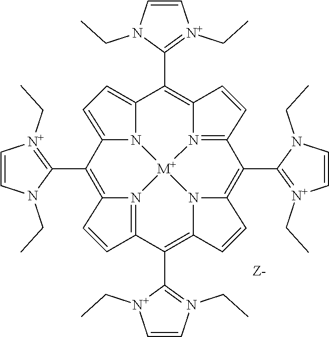

wherein Z− is a counterion.

In some embodiments, the meso-substituted metalloporphyrin is an alkyl substituted pyridyl porphyrin. Exemplary alkyl substituted pyridyl porphyrins include, but are not limited to, compounds having a structure of Formula B1 or B2:

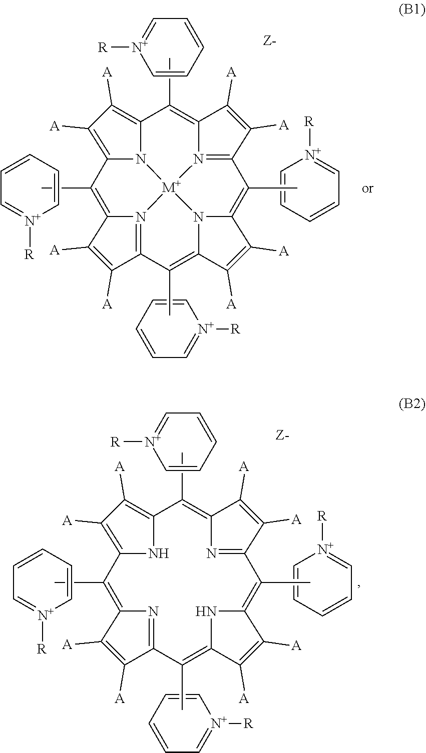

wherein:
each R is, independently, a C₁₋₁₂ alkyl (straight chain or branched), more preferably C₂₋₆ alkyl, and most preferably ethyl, propyl, butyl, or pentyl (straight chain or branched);
each A is, independently, a hydrogen or an electron withdrawing group (e.g., halogen, —NO₂ or —CHO);
M is metal selected from the group consisting of manganese, iron, copper, cobalt, nickel and zinc; and
Z⁻ is a counterion.

In some embodiments, the meso-substituted metalloporphyrin has a structure of Formula V:

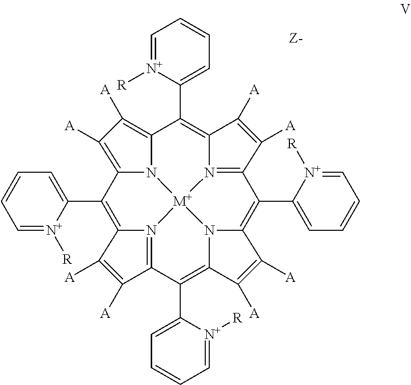

wherein each R, A, M, and Z is as given in connection with Formula B1 and B2 above.

In some embodiments, the meso-substituted metalloporphyrin has the structure:

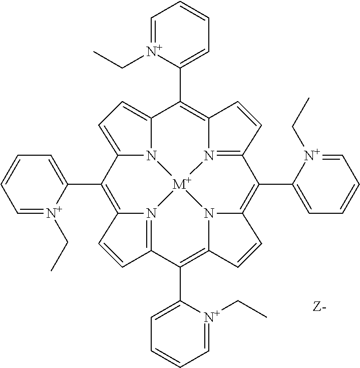

wherein Z⁻ is a counterion.

In some embodiments, the meso-substituted metalloporphyrin is an alkoxyalkyl substituted pyridyl porphyrin. Exemplary alkoxyalkyl substituted pyridyl porphyrins include, but are not limited to, those having a structure of Formula C1 or C2:

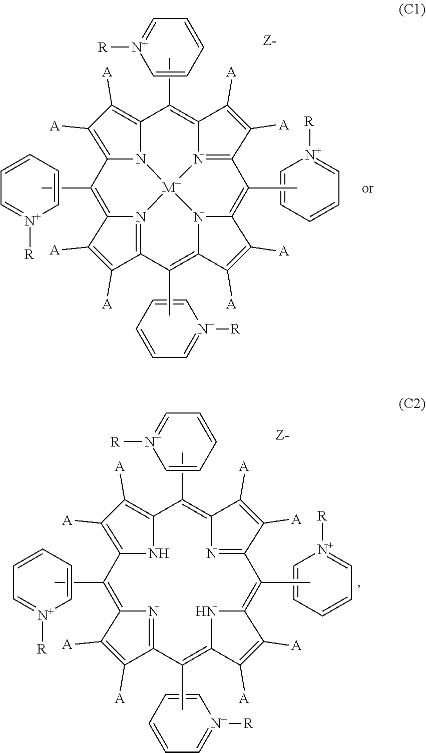

wherein:
each R is —(CH₂)ₘCH₂OX;
m is 1 or 2, preferably 1;
each X is, independently, a C₁₋₁₂ alkyl (straight chain or branched), more preferably C₂₋₆ alkyl, and most preferably ethyl, propyl, butyl, or pentyl (straight chain or branched);
each A is, independently, a hydrogen or an electron withdrawing group (e.g., halogen, NO₂ or CHO);
M is metal selected from the group consisting of manganese, iron, copper, cobalt, nickel and zinc; and
Z⁻ is a counterion.

In some embodiments, the meso-substituted metalloporphyrin has a structure of Formula V:

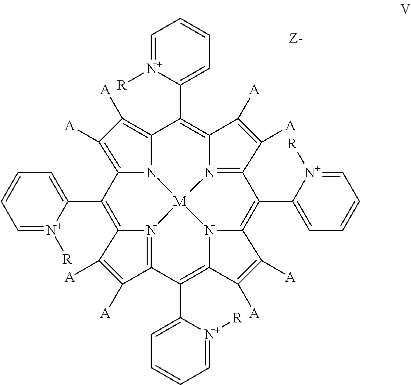

wherein each R, A, M, and Z is as given in connection with Formula C1 and C2 above.

In some embodiments, the meso-substituted metalloporphyrin has the structure:

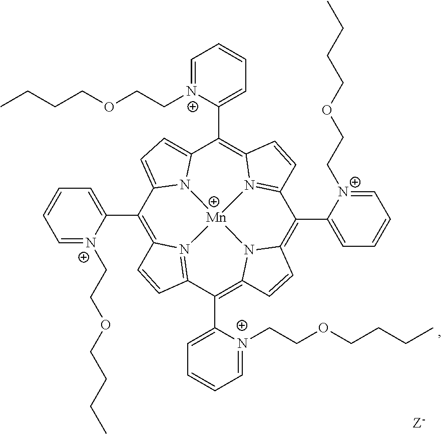

wherein Z⁻ is a counterion.

The meso-substituted metalloporphyrins disclosed herein can, as noted above, be prepared in the form of their salts or pharmaceutically acceptable salts, e.g., to provide a compound or composition including a counterion as noted above. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and/or (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

In some embodiments, the meso-substituted metalloporphyrin is MnTE-2-PyP⁵⁺ and has the structure:

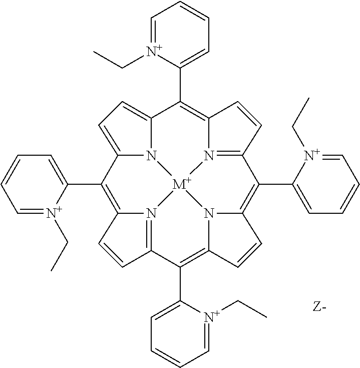

wherein M⁺ is manganese and Z⁻ is a counterion (e.g., an anion such as a halogen ion (e.g., chloride, fluoride, bromide, and/or iodide ion) and/or a conjugate base of an acid (e.g., an oleate)). In some embodiments, MnTE-2-PyP⁵⁺ in a composition of the present invention may comprise a halogen ion (e.g., chloride ion) as Z⁻ and/or a conjugate base of an acid (e.g., an oleate) as Z. In some embodiments, the meso-substituted metalloporphyrin is BMX-010, which has a structure of Formula VI:

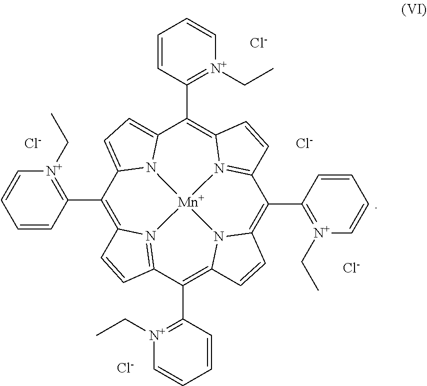

In some embodiments, a hydrophilic active ingredient of the present invention may be a fluoro-substituted porphyrin. A fluoro-substituted porphyrin of the present invention may have a structure represented by Formula VII:

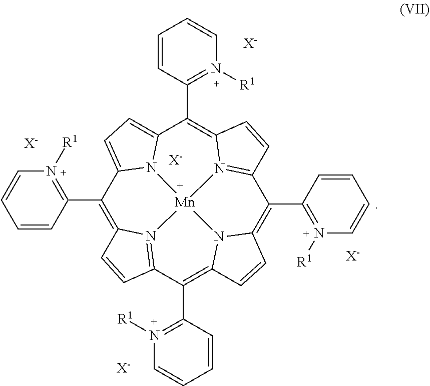

wherein:
R¹ is a C1-C8 alkyl that is substituted with at least 1 fluorine (e.g., a C1-C8 alkyl substituted with, e.g., 1, 2, 3, 4, 5, 6, 7, 8, 9, 10, 11, 12, 13, 14, 15, 16, or 17 fluorine atoms); and
X is a counterion (e.g., an anion such as a halogen ion (e.g., chloride, fluoride, bromide, and/or iodide ion) and/or a conjugate base of an acid (e.g., an oleate), PF₆, tosylate, besylate, and/or mesylate).

In some embodiments, in Formula VII, R¹ is a C1, C2, C3, C4, C5, C6, C7, or C8 alkyl group that is substituted with 1 to Y fluorine atoms, where Y is determined by the number of carbon atoms in the alkyl group times 2 plus 1. Thus, when R¹ in Formula VII is a C2 alkyl group, then it may be substituted with 1, 2, 3, 4, or 5 fluorine atoms (e.g., 2×2=4+1=5).

In some embodiments, a fluoro-substituted porphyrin of the present invention has a structure represented by Formula VIIa, Formula VIIb, Formula VIIc, Formula VIId, Formula VIIe, Formula VIIf, Formula VIIg, Formula VIIh, or Formula VIIi:

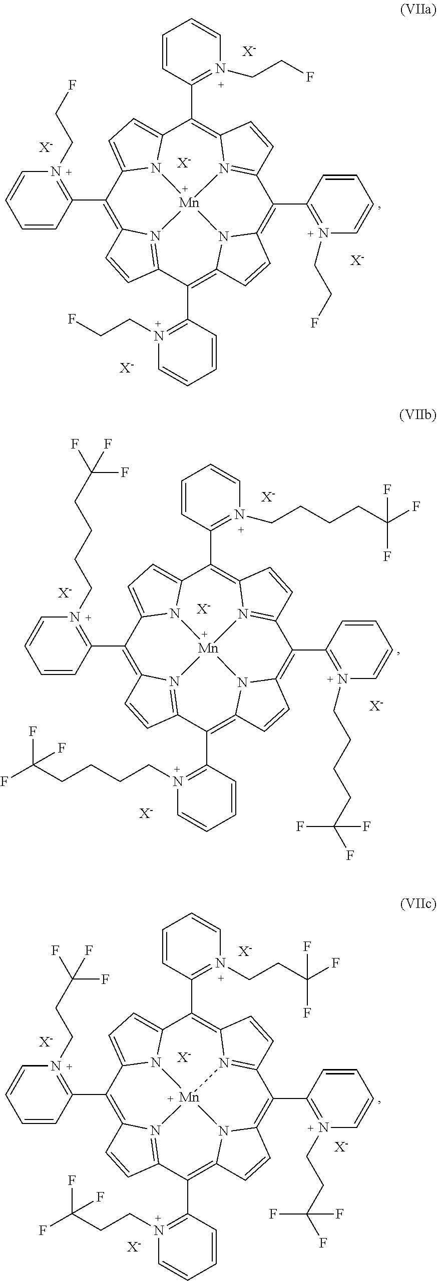

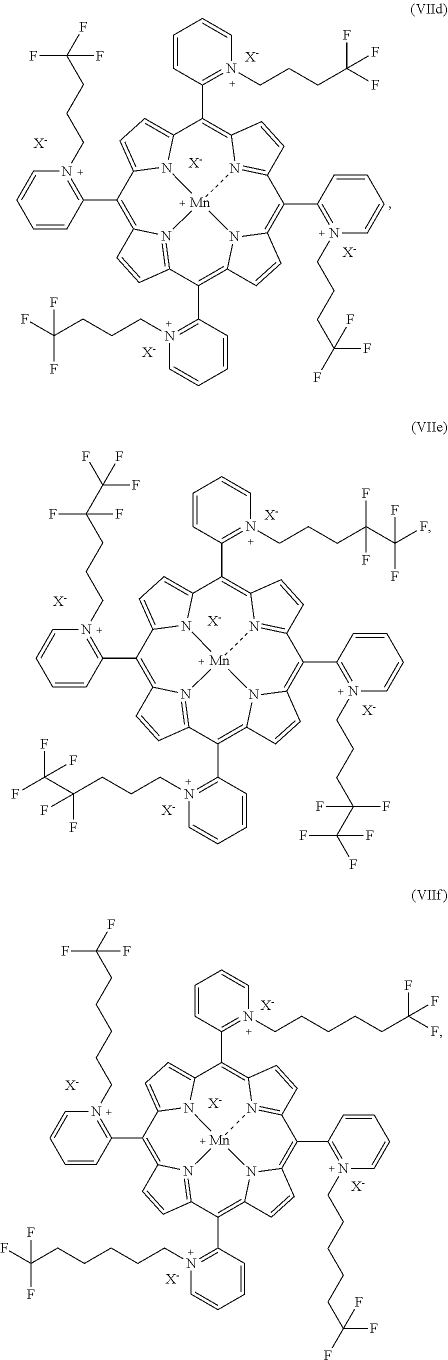

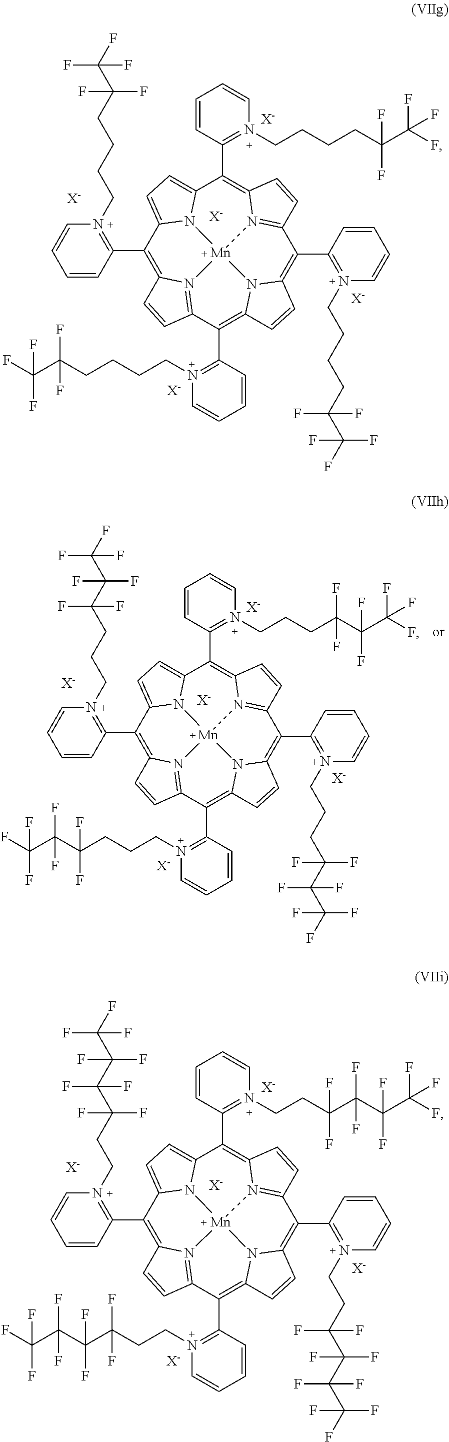

wherein X is a counterion as described herein (e.g., an anion such as a halogen ion (e.g., chloride, fluoride, bromide, and/or iodide ion) and/or a conjugate base of an acid (e.g., an oleate)).

A hydrophilic active ingredient may present in a composition of the present invention in an amount of about 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, or 0.9% to about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of the composition. In some embodiments, a hydrophilic active ingredient may present in a composition of the present invention in an amount of about 0.005%, 0.01%, 0.02%, 0.03%, 0.05%, 0.06%, 0.07%, 0.08%, 0.09%, 0.1%, 0.2%, 0.3%, 0.4%, 0.5%, 0.6%, 0.7%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of the composition. In some embodiments, a meso-substituted metalloporphyrin may be present in a composition of the present invention at a concentration in a range of about 0.005% 0.01%, 0.05%, or 0.1% to about 0.5%, 1%, 2%, 5%, or 10% by weight of the composition times the activity equivalent of BMX-010. Thus, when the meso-substituted metalloporphyrin is BMX-010, the meso-substituted metalloporphyrin may be present in the composition at a concentration in a range of about 0.005% 0.01%, 0.05%, or 0.1% to about 0.5%, 1%, 2%, 5%, or 10% by weight of the composition, respectively. When referring to the activity equivalent of BMX-010, activity equivalent is determined according to methods known to those of skill, such as, but not limited to, determined using a cytochrome c assay and/or pulse radiolysis (Spasojevic, Ivan, et al., Inorganic Chemistry, Vol. 40, No. 4, 2001, 726-739). For BMX-010, the calculated activity is 7676 units/mg by the cytochrome C assay. Accordingly, a dose of a meso-substituted metalloporphyrin (e.g., an Ames negative meso-substituted metalloporphyrin) that is equivalent to 0.01-0.1% of BMX-010 will have 767.6 to 7676 activity units per ml of the composition. When the meso-substituted metalloporphyrin is BMX-010, the composition may be administered to the subject with the meso-substituted metalloporphyrin present in an amount of about 0.005% to about 10% by weight of the composition. Accordingly, the activity equivalent of BMX-010 is 1 when the meso-substituted metalloporphyrin is BMX-010. Alternatively, when a meso-substituted metalloporphyrin is 20% less active than BMX-010, the activity equivalent of BMX-010 is 1.25, so the dosage may be determined by taking 0.005% times 1.25 to get the lower range concentration and by taking 10% times 1.25 to get the upper concentration. Thus, the concentration range for the meso-substituted metalloporphyrin that is 20% less active than BMX-010 is about 0.00625% to about 12.5% by weight of the composition.

In some embodiments, a hydrophilic active ingredient is micronized prior to inclusion in a composition of the present invention, optionally by jet milling, ball milling, spray drying, controlled crystallization, and/or other methods of producing particles. In some embodiments, a hydrophilic active ingredient is micronized to provide a plurality of particles in which at least about 90% of the particles in the plurality of particles have at least one dimension (e.g., diameter) in the micrometer range. In some embodiments, a hydrophilic active ingredient is micronized to provide a plurality of particles in which at least about 90% of the particles in the plurality of particles have at least one dimension (e.g., diameter) of about 50 microns or less. In some embodiments, a hydrophilic active ingredient, prior to inclusion in a composition of the present invention and/or in a composition of the present invention, has a D90 particle size of less than 50 microns, optionally a D90 of about 45, 40, or 30 microns. In some embodiments, a hydrophilic active ingredient, prior to inclusion in a composition of the present invention and/or in a composition of the present invention, has a D50 particle size of about 10 microns to about 25 microns. In some embodiments, a hydrophilic active ingredient, prior to inclusion in a composition of the present invention and/or in a composition of the present invention, has a D10 particle size of about 3, 3.5, or 4 microns to about 4.5, 5, 5.5, or 6 microns. In some embodiments, a hydrophilic active ingredient, prior to inclusion in a composition of the present invention and/or in a composition of the present invention, has a D90 particle size of less than 3 microns, optionally less than 2 microns. In some embodiments, a hydrophilic active ingredient, prior to inclusion in a composition of the present invention and/or in a composition of the present invention, has a D50 of about 0.5 or 0.75 microns to about 1, 1.5, 2, 2.5 or 3 micron(s). In some embodiments, a hydrophilic active ingredient, prior to inclusion in a composition of the present invention and/or in a composition of the present invention, has a D10 particle size of about 0.1, 0.2, or 0.3 microns to about 0.4, 0.5, or 0.6 microns. In some embodiments, a hydrophilic active ingredient, prior to inclusion in a composition of the present invention and/or in a composition of the present invention, has a D10 of about 0.1, 0.2, or 0.3 microns to about 0.4, 0.5, or 0.6 microns, a D50 of about 0.5, 0.75, 1, 1.5, 2, 2.5 or 3 micron(s), and/or a D90 of about 1 or 1.5 micron(s) to about 2 or 2.5 microns. In some embodiments, a hydrophilic active ingredient, prior to inclusion in a composition of the present invention and/or in a composition of the present invention, has a median particle size of about 1, 2, 3, or 4 micron(s) to about 5, 6, 7, 8, 9, 10, 11, 12 13, 14, or 15 microns. In some embodiments, a hydrophilic active ingredient, prior to inclusion in a composition of the present invention and/or in a composition of the present invention, has a median particle size of less than about 5 microns.

A composition of the present invention may comprise a hydrophilic active ingredient, a hydrophobic carrier, and an acid. In some embodiments, a composition of the present invention comprises a hydrophilic active ingredient, a hydrophobic carrier (e.g., a hydrocarbon carrier), a stiffening agent (e.g., a wax), and an acid. In some embodiments, a composition of the present invention comprises a hydrophilic active ingredient, a hydrophobic carrier (e.g., a hydrocarbon carrier), an oil, a stiffening agent (e.g., a wax), and an acid. In some embodiments, a hydrophilic active ingredient is dispersed and/or suspended in a composition of the present invention. In some embodiments, the hydrophobic carrier, stiffening agent, oil, and/or acid present in a composition of the present invention have a Hildebrand solubility parameter (δ) of less than 26 MPa^{1/2}. In some embodiments, the hydrophobic carrier, stiffening agent, oil, and/or acid independently have a Hildebrand solubility parameter (δ) of about 1, 2, 3, 4, 5, 6, 7, 8, 9, 10, 11, 12, 13, 14, 15, 16, 17, 18, 19, 20, 21, 22, 23, 24, 25, or 26 MPa^{1/2}. In some embodiments, the hydrophobic carrier, stiffening agent, oil, and/or acid independently have a Hildebrand solubility parameter (δ) of about 1, 5, or 10 to about 15, 20, or 26 MPa^{1/2}. A composition of the present invention may have a single phase and/or may be homogenous.

Exemplary acids that may be present in a composition of the present invention include, but are not limited to, oleic acid, adipic acid, benzoic acid, myristic acid, ricinoleic acid, stearic acid, coconut acid, isostearic acid, propionic acid, succinic acid, undecylenic acid, arachidonic acid, behenic acid, capric acid, caprylic acid, corn acid, cottonseed acid, dilinoleic acid, erucic acid, glycyrrhizic acid, lanolin acid, auric acid, methyl myristic acid, olive acid, palmitic acid, sebacic acid, and any combination thereof. In some embodiments, an acid present in a composition of the present invention is an organic acid. One or more acid(s) (e.g., 1, 2, 3, 4, or more) may be present in a composition of the present invention. In some embodiments, a composition of the present invention comprises oleic acid. In some embodiments, an acid present in a composition of the present invention may ion pair with a hydrophilic active ingredient present in the composition.

A composition of the present invention may comprise an acid (e.g., oleic acid) in an amount of about 0.001%, 0.005%, 0.01%, 0.05%, or 0.1% to about 0.2%, 0.5%, 1%, 2%, or 2.5% by weight of the composition. In some embodiments, an acid (e.g., oleic acid) is present in a composition of the present invention in an amount of about 0.001%, 0.005%, 0.01%, 0.1%, 0.2%, 0.5%, 1%, 2%, or 2.5% by weight of the composition. In some embodiments, a composition of the present invention comprises an acid (e.g., oleic acid) in an amount of about 0.15% or less by weight of the composition such as in an amount of about 0.005%, or 0.01% to about 0.05%, 0.1%, or 0.15% by weight of the composition. In some embodiments, a composition of the present invention comprises an acid (e.g., oleic acid) in an amount of about 0.001%, 0.002%, 0.003%, 0.004%, 0.005%, 0.006%, 0.007%, 0.008%, 0.01%, 0.02%, 0.03%, 0.04%, 0.05%, 0.06%, 0.07%, 0.08%, 0.09%, 0.1%, or 0.15% by weight of the composition. In some embodiments, an acid is solubilized and/or dissolved in a composition of the present invention. In some embodiments, an acid present in a composition of the present invention has a pKa of about 3, 3.5, 4, 4.5, 5, or 5.5. In some embodiments, an acid present in a composition of the present invention has a pKa in a range of about 3, 3.5, or about 4, 4.5, 5, or 5.5.

In some embodiments, a component present in a composition of the present invention (e.g., an acid) has a Hydrophile Lipophile Balance (HLB) of 10 or less such as about 0.5, 1, 2, 3, 4, 5, 6, 7, 8, 9, or 10. In some embodiments, a component present in a composition of the present invention (e.g., an acid) has a HLB of about 5 or less such as about 5, 4.5, 4, 3.5, 3, 2.5, 2, 1.5, 1, 0.5, or less. In some embodiments, a component present in a composition of the present invention (e.g., an acid) has a HLB of about 1. In some embodiments, a component present in a composition of the present invention (e.g., an acid) has a HLB in a range of about or 1 to about 1.5, 2, 3, 4, 5, 6, 7, 8, 9, or 10.

Exemplary hydrophobic carriers that may be present in a composition of the present invention include, but are not limited to, a petrolatum such as white petrolatum (e.g., white petrolatum NF and/or USP) a semisolid mixture of hydrocarbons (e.g., a semisolid mixture of branched and/or unbranched hydrocarbon chains, aromatic hydrocarbons, and/or cyclic alkanes, which may be similar and/or comparable to a petrolatum), a semipolar hydrocarbon (e.g., medium chain triglycerides, coconut oil, and/or beeswax), a stiffening agent (e.g., a wax), and/or an oil. One or more hydrophobic carrier(s) (e.g., 1, 2, 3, 4, or more) may be present in a composition of the present invention. In some embodiments, a hydrophobic carrier comprises one or more (e.g., 1, 2, 3, 4, or more) hydrocarbons. In some embodiments, a hydrophobic carrier comprises a hydrocarbon carrier (e.g., a petrolatum), a stiffening agent (e.g., a wax), and optionally an oil. In some embodiments, a composition of the present invention comprises a petrolatum, optionally white petrolatum. In some embodiments, a hydrophobic carrier is present in a composition of the present invention in an amount of about 1%, 5%, 10%, 15%, 20%, 25%, 30%, 35%, 40%, or 45% to about 50%, 55%, 60%, 65%, 70%, 75%, 80%, 85%, 90%, 95%, 98%, or 99% by weight of the composition. In some embodiments, a hydrophobic carrier (e.g., a hydrocarbon carrier such as petrolatum) is present in a composition of the present invention in an amount of about 60%, 65%, 70%, or 75% to about 80%, 85%, 90%, 95%, 98%, or 99% by weight of the composition. In some embodiments, a hydrophobic carrier (e.g., a hydrocarbon carrier such as petrolatum) is present in a composition of the present invention in an amount of about 60%, 65%, 70%, 75%, 80%, 85%, 90%, 95%, or 98% by weight of the composition. In some embodiments, a hydrophobic carrier such as a hydrocarbon carrier (e.g., prior to inclusion in a composition of the present invention) is a solid or semisolid at room temperature. A hydrophobic carrier (e.g., a hydrocarbon carrier such as petrolatum) present and/or used in a composition of the present invention may have a melting point in a range of about 30° C., 35° C., 40° C., 45° C., or 50° C. to about 55° C., 60° C., 65° C., 70° C., 75° C., or 80° C. In some embodiments, a hydrophobic carrier present and/or used in a composition of the present invention may have a melting point of about 30° C., 35° C., 40° C., 45° C., 50° C., 55° C., 60° C., 65° C., 75° C., or 80° C.

Exemplary stiffening agents that may be present in a composition of the present invention include, but are not limited to, orange wax, lanolin, cetyl esters wax, synthetic sperm whale wax, paraffin, bayberry wax, beeswax (e.g., white and/or yellow beeswax), ozokerite wax, carnauba wax, castor wax, glyceryl monostearate, propylene glycol stearate, and/or microcrystalline wax. In some embodiments a composition of the present invention comprises a wax. In some embodiments, a composition of the present invention comprises a paraffin (e.g., paraffin NF). In some embodiments, a composition of the present invention comprises a microcrystalline wax. In some embodiments, a macrocrystalline wax is used to prepare a composition of the present invention. A stiffening agent present and/or used in a composition of the present invention may have a melting point in a range of about 35° C., 40° C., 45° C., or to about 55° C., 60° C., 65° C., 70° C., 75° C., 80° C., 85° C., 90° C., 95° C., 100° C., or 105° C. In some embodiments, a stiffening agent present and/or used in a composition of the present invention may have a melting point of about 35° C., 40° C., 45° C., 50° C., 55° C., 60° C., 65° C., 70° C., 80° C., 85° C., 90° C., 95° C., 100° C., or 105° C. In some embodiments, a stiffening agent present and/or used in a composition of the present invention may have a melting point in a range of about 50° C., 51° C., 52° C., 53° C., or 54° C. to about 55° C., 56° C., 57° C., 58° C., 59° C., or In some embodiments, a stiffening agent present and/or used in a composition of the present invention may have a melting point in a range of about 55° C., 56° C., 57° C., 58° C., or 59° C. to about 60° C., 61° C., 62° C., 63° C., 64° C., or 65° C. In some embodiments, a composition of the present invention comprises a stiffening agent in an amount of about 0.5%, 1%, 1.5%, 2%, 2.5%, 3%, 3.5%, 4%, or 4.5% to about 5%, 5.5%, 6%, 6.5%, 7%, 7.5%, 8%, 8.5%, 9%, 9.5%, or 10% by weight of the composition. In some embodiments, a composition of the present invention comprises a stiffening agent in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of the composition. In some embodiments, a stiffening agent (e.g., prior to inclusion in a composition of the present invention) is a solid at room temperature. In some embodiments, a stiffening agent has a low solubility in oil and needs to be heated in order to provide a single phase and/or to solubilize the wax in the oil.

Exemplary oils that may be present in a composition of the present invention include, but are not limited to, a coconut oil, mineral oil (e.g., a mineral oil USP and/or NF and/or light mineral oil), soybean oil, olive oil, isopropyl myristate, and/or a composition comprises one or more medium chain triglyceride(s). One or more oil(s) (e.g., 1, 2, 3, 4, or more) may be present in a composition of the present invention. In some embodiments, a composition of the present invention comprises a mineral oil (e.g., a mineral oil USP and/or NF). In some embodiments, an oil is present in a composition of the present invention in an amount of about 1%, 5%, or 10% to about 15%, 20%, or 25% by weight of the composition. In some embodiments, an oil is present in a composition of the present invention in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% by weight of the composition. In some embodiments, an oil (e.g., prior to inclusion in a composition of the present invention) is present in a composition of the present invention in an amount of less than about 15% by weight of the composition. In some embodiments, an oil is present in a composition of the present invention in an amount in a range of about 1%, 2%, 3%, 4%, 5%, or 6% to about 7%, 8%, 9%, 10%, 11%, 12%, 13%, 14%, or 15% by weight of the composition. In some embodiments, an oil is a liquid at room temperature. In some embodiments, an oil has a kinematic viscosity in a range of about 1, 2, 3, 4, 5, 10, 15, 20, or 25 mm²·s⁻¹ to about 30, 40, 50, 60, 70, 80, 90, 100, 110, 120, 130, 140, 150, 160, or 170 mm²·s⁻¹, optionally as measured with a capillary viscometer at 40±0.1°.

In some embodiments, the hydrophobic carrier, stiffening agent (e.g., a wax), oil, and/or acid is non-polar. In some embodiments, a composition of the present invention comprises a single polar compound, optionally wherein the single polar compound is the hydrophilic active ingredient and each of the remaining components in the composition is nonpolar. In some embodiments, a composition of the present invention is devoid of a hydrophilic component (e.g., a hydrophilic compound) other than the hydrophilic active ingredient (e.g., the composition is devoid an additional or second hydrophilic component). A composition of the present invention may be nonaqueous and/or may not include water as an added component. In some embodiments, the hydrophobic carrier, stiffening agent, oil, and/or acid include minimal or no water (e.g., are not hygroscopic). In some embodiments, a composition of the present invention is not hygroscopic. In some embodiments, a composition of the present invention and/or a hydrophobic carrier, stiffening agent, oil, and/or acid may include water in an amount of about 0, 5, 10, 50, 100, or 200 ppm to about 300, 400, 500, or 600 ppm such as about 5, 10, 50, 100, 200, 300, 400, 500, or 600 ppm. In some embodiments, water may be present in a composition of the present invention in an amount of about 0%, 0.1%, or 0.5% to about 1%, 2%, 3%, 4%, or 5% by weight of the composition. In some embodiments, water may be present in a composition of the present invention in an amount of about 0%, 0.1%, 1%, 2%, 3%, 4%, or 5% by weight of the composition.

A composition of the present invention may have a water activity of less than about such as a water activity of about 0.7, 0.65, 0.6, 0.55, 0.5, 0.45, 0.4, 0.35, 0.3, 0.25, 0.2, 0.1, or less. In some embodiments, a composition of the present invention has water activity of less than about 0.4. In some embodiments, a composition of the present invention has water activity of about 0.1 or 0.2 to about 0.3 or 0.4.

In some embodiments, a composition of the present invention is devoid of a polyhydric alcohol (e.g., a glycol such as propylene glycol), a fatty alcohol (e.g., cetostearyl alcohol), a fatty acid ester (e.g., isopropyl palmitate and/or a polyoxyethylene sorbitol ester), and/or a cellulose (e.g., hydroxyethyl cellulose, hydroxymethyl cellulose, etc.). In some embodiments, a composition of the present invention is devoid of propylene glycol, cetostearyl alcohol, isopropyl palmitate, titanium dioxide, a polyoxyethylene sorbitol ester, and/or a hydroxyethyl cellulose. In some embodiments, a composition of the present invention is not a gel (e.g., is not an aqueous gel) and/or is not a cream. In some embodiments, a composition of the present invention is devoid of a preservative and/or is devoid of an antimicrobial.

In some embodiments, a composition of the present invention comprises a hydrophilic active ingredient (e.g., a meso-substituted metalloporphyrin) in an amount of about 0.005%, 0.05%, 0.1%, or 0.5% to about 1%, 2%, 5%, or 10% by weight of the composition, a hydrophobic carrier (e.g., a petrolatum) in an amount of about 60% to about 99% by weight of the composition, a stiffening agent (e.g., a paraffin) in an amount of about 1% to about 10% by weight of the composition, and an acid in an amount of about 0.001%, 0.005%, 0.01%, 0.05%, or 0.1% to about 0.2%, 0.5%, 1%, 2%, or 2.5% by weight of the composition. In some embodiments, a composition of the present invention comprises a hydrophilic active ingredient (e.g., a meso-substituted metalloporphyrin) in an amount of about 0.005%, 0.01%, 0.05%, or 0.5% to about 1%, 2%, 5%, or 10% by weight of the composition, a hydrophobic carrier (e.g., a petrolatum) in an amount of about 60% to about 99% by weight of the composition, a stiffening agent (e.g., a paraffin) in an amount of about 1% to about 10% by weight of the composition, an acid in an amount of about 0.001%, 0.005%, 0.01%, 0.05%, or to about 0.2%, 0.5%, 1%, 2%, or 2.5% by weight of the composition, and an oil (e.g., a mineral oil) in an amount of about 5% to about 25% by weight of the composition. In some embodiments, a composition of the present invention comprises a meso-substituted metalloporphyrin (e.g., BMX-010), a petrolatum, a paraffin wax, a mineral oil, and oleic acid. In some embodiments, a composition of the present invention comprises a meso-substituted metalloporphyrin (e.g., BMX-010), a petrolatum, a paraffin wax, and oleic acid.

One or more (e.g., 1, 2, 3, 4, 5, or more) excipient(s) may be present in a composition of the present invention. In some embodiments, a composition of the present invention comprises a colorant and/or a color masking agent such as, but not limited to, titanium dioxide, zinc oxide, and/or micronized polymers and/or waxes (e.g., Teflon®, polypropylene, polyethylene, and/or carnauba wax). In some embodiments, a composition of the present invention is devoid of a colorant and/or a color masking agent. In some embodiments, a composition of the present invention is devoid of a colorant and/or a color masking agent that is not a wax. In some embodiments, a composition of the present invention comprises a colorant and/or color masking agent that is a wax that is not melted.

In some embodiments, a composition of the present invention is devoid of a penetration enhancing agent (e.g., devoid of an agent that increases permeation and/or penetration of a hydrophilic active ingredient across the stratum corneum upon topical administration to skin of a subject). In some embodiments, to the extent an acid, hydrophobic carrier, stiffening agent, and/or oil in a composition of the present invention may be deemed a penetration enhancing agent, then the composition is devoid of an additional penetration enhancing agent. In some embodiments, a composition of the present invention is devoid of a penetration enhancing agent in an amount of greater than about 0.5%, 1%, 2%, or 3% by weight of the composition. In some embodiments, a composition of the present invention is devoid of squalene, cyclomethicone, and/or oleyl alcohol.

In some embodiments, a composition of the present invention comprises an unsaturated fatty alcohol such as, but not limited to, oleyl alcohol, linoleyl alcohol, and/or linolenyl alcohol. A composition of the present invention may comprise an unsaturated fatty alcohol (e.g., oleyl alcohol) in an amount of about 0.005%, 0.01%, 0.05%, or 0.1% to about 0.2%, 0.5%, 1%, 2%, 3%, 4%, 5%, 6%, 7%, 8%, 9%, or 10% by weight of the composition. In some embodiments, an unsaturated fatty alcohol (e.g., oleyl alcohol) is present in a composition of the present invention in an amount of about 0.005%, 0.01%, 0.05%, 0.1%, 0.2%, 0.5%, 1%, 2%, 3%, 4%, 5%, 6%, 7%, 8%, 9%, or 10% by weight of the composition. In some embodiments, a composition of the present invention is devoid of unsaturated fatty alcohol (e.g., oleyl alcohol). In some embodiments, a composition of the present invention is an ointment. An “ointment” as used herein refers to a composition comprising one or more hydrophobic components (e.g., a hydrophobic carrier and/or oil) in an amount of greater than 50% by weight of the composition. In some embodiments, a composition of the present invention is an ointment that comprises a hydrocarbon base. In some embodiments, a composition of the present invention is devoid of low molecular weight branched hydrocarbons. In some embodiments, a composition and/or ointment of the present invention is a semisolid (e.g., the composition is not pourable at room temperature). In some embodiments, a composition and/or ointment of the present invention is a solid (e.g., in the form of a solid stick), optionally a solid that may be configured to glide across skin of a subject.

In some embodiments, a composition of the present invention is a solid (e.g., a solid stick) and the hardness of the composition may be measured using a needle or polished cone of a particular weight and dimension. The needle or polished cone may be allowed to travel downward through the composition for a predetermined period of time, and the distance traveled by the needle or cone may be a relative measure of the hardness of the composition. For example, hardness of a composition may be tested with a penetration cone (e.g., Model H1310; sold by Humboldt Manufacturing Company) weighing 2.52 grams, and a Precision Model 14AN-8 Penetrometer (sold by GCA Corp). In some embodiments, a composition of the present invention has a penetration value of about 1, 2, 3, 4, 5, 10, or 15 millimeter(s) to about 20, 30, 40, 50, 60, 70, or 80 millimeters over a period of 5 seconds.

In some embodiments, a composition of the present invention comprises a hydrophobic carrier, a stiffening agent, an acid, and/or an hydrophilic active ingredient (e.g., prior to inclusion in a composition of the present invention) that are each a solid at room temperature (e.g., about 20-25° C.) and/or an oil (e.g., prior to inclusion in a composition of the present invention) that is a liquid at room temperature.

A composition of the present invention may have a viscosity in a range of about 15,000, 25,000, 30,000, 35,000, or 40,000 to about 45,000, 50,000, 55,000, 60,000, 65,000, 75,000, 80,000, 85,000, 90,000, 95,000, or 100,000 cP, optionally as measured using a Brookfield HA DV-3T with the following parameters: Spindle: CPA-52Z, Test Speed: 5 RPM, Sample Size: ˜0.5 g, Temperature: 25° C.±0.1° C., Equilibration: 5 minutes, and Read Time: 2 minutes. In some embodiments, a composition of the present invention has a viscosity of about 15,000, 20,000, 25,000, 30,000, 35,000, 40,000, 45,000, 50,000, 55,000, 60,000, 70,000, 75,000, 80,000, 85,000, 90,000, 95,000, or 100,000 cP, optionally as measured using a Brookfield HA DV-3T with the following parameters: Spindle: CPA-52Z, Test Speed: RPM, Sample Size: ˜0.5 g, Temperature: 25° C.±0.1° C., Equilibration: 5 minutes, and Read Time: 2 minutes. In some embodiments, a composition of the present invention has a viscosity of about 25,000, 26,000, 27,000, 28,000, 29,000, 30,000, 31,000, 32,000, 33,000, 34,000, or cP, optionally as measured using a Brookfield HA DV-3T with the following parameters: Spindle: CPA-52Z, Test Speed: 5 RPM, Sample Size: ˜0.5 g, Temperature: 25° C.±0.1° C., Equilibration: 5 minutes, and Read Time: 2 minutes. Viscosity may be measured using methods known to those of skill in the art. In some embodiments, viscosity may be measured using a Brookfield HA DV-3T with the following parameters: Spindle: CPA-52Z, Test Speed: 5 RPM, Sample Size: ˜0.5 g, Temperature: 25° C.±0.1° C., Equilibration: 5 minutes, and Read Time: 2 minutes.

A composition of the present invention may have improved (e.g., increased) storage, uniformity, and/or stability and/or a composition of the present invention may have improved (e.g., increased) delivery (e.g., permeation) of a hydrophilic active ingredient. In some embodiments, a composition of the present invention does not separate after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity, optionally at about 25° C. or 30° C. with about 65% relative humidity. Separation of a composition of the present invention may be determined and/or measured visually (e.g., by human eye); for example, by no visible phase separation and/or by no visible change in physical appearance and/or texture of the composition.

In some embodiments, a composition of the present invention has improved uniformity after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity, optionally at about 25° C. or 30° C. with about 65% relative humidity. Uniformity of a composition of the present invention may be determined and/or measured by measuring the concentration of one or more component(s) of the composition at one or more position(s) within the composition and comparing the concentration of each of the one or more component(s) at each respective position of the one or more position(s) to a starting or initial concentration of the respective component (e.g., the amount of a component at initial formation of the composition) and/or to a desired concentration for the respective component (e.g., a concentration provided on a label for a commercial and/or approved product (e.g., a label claim)). For example, in some embodiments, uniformity may be determined and/or measured by measuring the concentration of a hydrophilic active ingredient present in the composition at the top (e.g., a portion of the composition that includes a surface of the composition exposed to air and/or that is adjacent to an opening (e.g., cap) of the container in which the composition is present), middle (e.g., a portion approximately equally distanced between the top and bottom portions), and bottom (e.g., a portion of the composition that includes a surface of the composition in contact with the container in which it is present (e.g., at the crimp of a tube)) portions of the composition after storage for a period of time and comparing the concentration of the hydrophilic active ingredient at each of the top, middle, and bottom portions to a starting or initial concentration of the hydrophilic active ingredient optionally at each of the top, middle and bottom portions or to a desired concentration of the hydrophilic active ingredient optionally at each of the portions. In some embodiments, the concentration of a hydrophilic active ingredient in a composition of the present at one or more position(s) (e.g., 1, 2, 3, 4, or more) within the composition after storage (e.g., storage for one or more month(s) at about 25° C. or 30° C. with about 65% relative humidity) is within about 85%, 90%, or 95% to about 100%, 105%, 110%, or 115% of a starting or initial concentration of the hydrophilic active ingredient optionally at the one or more position(s) or relative to a desired concentration of the hydrophilic active ingredient. In some embodiments, the concentration of a hydrophilic active ingredient in a composition of the present invention after storage at one or more position(s) (e.g., top, middle, and/or bottom portion(s)) of the composition is within about 85%, 90%, or 95% to about 100%, 105%, 110%, or 115% of a label claim for the amount of hydrophilic active ingredient present in the composition.

In some embodiments, a composition of the present invention has a low concentration of impurities (e.g., less than about 5%, 4%, 3%, 2%, or 1%) after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity, optionally at about 25° C. or 30° C. with about 65% relative humidity. In some embodiments, a composition of the present invention has a reduced concentration of impurities after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity, optionally at about 25° C. or 30° C. with about 65% relative humidity, compared to the amount of impurities present in a composition not in accordance with the present invention (e.g., a composition that is not an ointment such as in the form of a cream or gel). In some embodiments, a composition of the present invention improves the stability of a hydrophilic active ingredient present in the composition such as by providing less impurities over a period of time with certain storage conditions (e.g., storage for one or more month(s) at about 25° C. or 30° C. with about 65% relative humidity) than for the hydrophilic active ingredient alone or for the hydrophilic active ingredient present in a composition not in accordance with the present invention over the same period of time and storage conditions. In some embodiments, a composition of the present invention comprising a hydrophilic active ingredient has a reduced concentration of impurities after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity (e.g., at about 25° C. or 30° C. with about 65% relative humidity) compared to the amount of impurities present in after the same storage conditions for the hydrophilic active ingredient alone. Exemplary impurities include, but are not limited to, a degradation product of a hydrophilic active ingredient, a contaminant, and/or a component other than those added specifically to provide a composition of the present invention. The concentration and/or presence of impurities may be measured by methods known to those of skill in the art such as, but not limited to chromatography methods (e.g., liquid chromatography) and/or mass spectrometry.

In some embodiments, after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity (e.g., at about 25° C. or with about 65% relative humidity), a composition of the present invention has a viscosity that changes by about ±40%, 35%, 30%, 25%, 20%, or less compared to the viscosity of the composition at the time of formation of the composition and/or at the start of storage (e.g., the beginning of day 1 of the storage time period). In some embodiments, after storage, optionally in a closed container, for about 1, 2, 3, 4, 5, 6, 7, 8, 9, 10, 11, or 12 months or more at about or 30° C. to about 35° C. or 40° C. with about 50%, 55%, or 60% to about 70% or 75% relative humidity (e.g., at about 25° C. or 30° C. with about 65% relative humidity), a composition of the present invention has a viscosity that increases by about 40%, 35%, 30%, 25%, 20%, or less compared to the viscosity of the composition at the time of formation of the composition and/or at the start of storage (e.g., the beginning of day 1 of the storage time period).

In some embodiments, after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity (e.g., at about 25° C. or with about 65% relative humidity), the particle size (e.g., the D50) of a hydrophilic active ingredient present in a composition of the present invention changes by about ±50%, 45%, 40%, 35%, 30%, 25%, 20%, or less compared to the particle size of the hydrophilic active ingredient in the composition at the time of formation of the composition and/or at the start of storage (e.g., the beginning of day 1 of the storage time period). In some embodiments, after storage, optionally in a closed container, for about 1, 2, 3, 4, 5, 6, 7, 8, 9, 10, 11, or 12 months or more at about 20° C. or 30° C. to about 35° C. or 40° C. with about 50%, 55%, or 60% to about 70% or 75% relative humidity (e.g., at about 25° C. or 30° C. with about 65% relative humidity), the particle size (e.g., the D50) of a hydrophilic active ingredient present in a composition of the present invention increases by about ±50%, 45%, 40%, 35%, 30%, 25%, 20%, or less compared to the particle size of the hydrophilic active ingredient in the composition at the time of formation of the composition and/or at the start of storage (e.g., the beginning of day 1 of the storage time period).

In some embodiments, a composition of the present invention reduces oxidation of a hydrophilic active ingredient (e.g., BMX-010) present in the composition. In some embodiments, a composition of the present invention is devoid an antioxidant. Exemplary antioxidants include, but are not limited to, butylated hydroxyanisole (BHA), ethylenediaminetetraacetic acid (EDTA), and/or ascorbic acid. In some embodiments, a composition of the present invention comprises a hydrophilic active ingredient that is an antioxidant (e.g., BMX-010) and the composition is devoid an additional antioxidant (e.g., devoid of BHA, EDTA, and/or ascorbic acid).

A composition of the present invention may have improved (e.g., increased) delivery (e.g., permeation) of a hydrophilic active ingredient to a subject compared to a composition comprising the same hydrophilic active ingredient in the same amount but in a composition not in accordance with the present invention (e.g., the composition is a cream or a gel (e.g., an aqueous gel)). The amount of a hydrophilic active ingredient delivered to a subject may be determined with methods known to those of skill in the art. For example, in some embodiments, a Franz diffusion cell with mammalian skin (e.g., human skin such as human cadaver skin) may be used to measure and/or determine the amount of a hydrophilic active ingredient that is delivered to a subject. In some embodiments, a composition of the present invention may deliver a hydrophilic active ingredient that is present in the composition to the dermis and/or epidermis of a subject that is topically administered the composition. In some embodiments, a method of the present invention topically administers a composition of the present invention comprising a hydrophilic active ingredient in an amount in a range of about to about 0.5%, 1%, or 10% by weight of the composition to the dermis in an amount of about 5 or 10 nanograms per cm² to about 15, 20, 25, 30, 40, 45, 50, 55, 60, 65, 70, 75, 80, 85, 95, 100, 125, 150, 175, or 200 nanograms per cm² or more and/or to the epidermis in an amount of about 5 or 10 nanograms per cm² to about 15, 20, 25, 30, 40, 45, 50, 55, 60, 65, 70, 90, 100, 110, 120, 130, 140, 150, 160, 170, 180, 190, 200, 300, 400, 500, 600, 700, 800, 900, 1000, 1100, 1200, 1300, 1400, or 1500 nanograms per cm² or more, optionally as measured using Franz diffusion cells with human cadaver skin. In some embodiments, a composition of the present invention may deliver more of the hydrophilic active ingredient to the epidermis than the dermis. In some embodiments, a composition of the present invention may deliver a hydrophilic active ingredient to the dermis in an amount of at least about 20, 30, or 50 ng/cm² of the dermis and/or may deliver a hydrophilic active ingredient to the epidermis in an amount of at least about 400 or 500 ng/cm² of the epidermis. In some embodiments, a composition of the present invention may deliver and/or administer a hydrophilic active ingredient to the epidermis and/or dermis of a subject in an amount that is about 0.05% or 0.1% to about 0.25% or 0.5% of the amount of the hydrophilic active ingredient present in the composition administered to the skin of the subject. For example, a composition of the present invention comprising 0.1% of a hydrophilic active ingredient by weight of the composition may administer to the dermis and/or epidermis about 0.005% to about 0.05% of the hydrophilic active ingredient by weight of the composition.

In some embodiments, a composition of the present invention may be compared to a composition not in accordance with the present invention such as a gel having a composition as provided in Table 1 and/or a cream as provided in Table 2.

TABLE 1
BMX-010 0.01%, 0.03%, and 0.1% gel formulations that are not in accordance with
the present invention.

| | Quantity per batch, g | | | |
|---|---|---|---|---|
| | (wt %) | | | |
| Component, Quality | | 0.01% | 0.03% | 0.1% |
| (Function) | Placebo | BMX-010 | BMX-010 | BMX-010 |
| BMX-010, AMRI | — | 1 | 3 | 10 |
| (API) | | (0.01%) | (0.03%) | (0.10%) |
| Natrosol ™ Hydroxyethylcellulose | 300 | 300 | 300 | 300 |
| HX, Pharm, NF | (3.00%) | (3.00%) | (3.00%) | (3.00%) |
| (gelling agent) | | | | |
| Propylene Glycol, USP | 500 | 500 | 500 | 500 |
| (gelling agent) | (5.00%) | (6.00%) | (5.00%) | (5.00%) |
| Potassium Sorbate Powder, NF | 15 | 15 | 15 | 15 |
| (preservative) | (0.15%) | (0.15%) | (0.15%) | (0.15%) |
| Sodium Benzoate, NF | 30 | 30 | 30 | 30 |
| (preservative) | (0.30%) | (0.30%) | (0.30%) | (0.30%) |
| Titanium Dioxide, USP | 200 | 200 | 200 | 200 |
| (colorant) | (2.00%) | (2.00%) | (2.00%) | (2.00%) |
| Glacial Acetic Acid, USP | 15.2 | 15.2 | 15.2 | 15.2 |
| (buffer) | (0.15%) | (0.15%) | (0.15%) | (0.15%) |
| Sodium Hydroxide 1N solution, USP | 184.4 | 164.4 | 164.4 | 164.4 |
| (buffer) | (1.64%) | (1.64%) | (1.64%) | (1.64%) |
| Sterile Purified Water, USP | 8775.4 | 8774.4 | 8772.4 | 8765.4 |
| (solvent) | (87.75%) | (87.74%) | (87.72%) | (87.65%) |
| Medical Air, USP | 0 | 0 | 0 | 0 |
| (blowing agent) | | | | |
| Total batch weight | 10000 | 10000 | 10000 | 10000 |

TABLE 2
Cream base formulation not in accordance with the present invention
that can include 0.03% or 0.1% BMX-010 by weight of the formulation.

| Ingredient | Amount |
|---|---|
| 25 mM Acetate pH 5.0 | quantity sufficient to total 100% by weight |
| Mineral Oil | 12.0% |
| Petrolatum | 9.0% |
| Cetostearyl alcohol | 12.0% |
| Propylene glycol | 8.0% |
| Tween 20 | 1.0% |
| Isopropyl palmitate | 1.0% |
| Imidazolidinyl urea | 0.2% |
| Methylparaben | 0.2% |
| Propylparaben | 0.1% |
| BMX-010 | 0.03%-0.1% |

A composition of the present invention may have a color, prior to, during, and/or after administration to a subject (e.g., prior to, during, and/or after application onto skin of a subject), that is clear, translucent, white, and/or off-white. In some embodiments, a composition of the present invention, prior to, during, and/or after administration to a subject (e.g., prior to, during, and/or after application onto skin of a subject), has an ASTM D1500 color of less than about 1.0 such as about 0.9, 0.8, 0.7, 0.6, 0.5, 0.4, 0.3, 0.2, 0.1 or less, optionally as measured in accordance with ASTM D1500, and/or a color on the Saybolt Color Scale in a range of about +30 to about −16, optionally as measured in accordance for ASTM D156. In some embodiments, a composition of the present invention, prior to, during, and/or after administration to a subject (e.g., prior to, during, and/or after application onto skin of a subject), has a color of less than about 0.5 (e.g., about 0.4, 0.3, 0.2, 0.1 or less), optionally as measured in accordance with ASTM D1500, and/or a color on the Saybolt Color Scale in a range of about +30 or +25 to about 0 or −5, optionally as measured in accordance for ASTM D156. In some embodiments, a composition of the present invention has a color, as determined by the CIE L*a*b* system using a Hunter Labs Colorflex instrument with D65/10° illumination, with a L* value in a range of about 5 or 10 to about 20, a* value in a range of about 5 to about 15 or 20, and a b* value of about −5 or 0.5 to about 5. In some embodiments, a composition of the present invention comprising BMX-010 in an amount of 0.1% by weight of the composition has a color, as determined by the CIE L*a*b* system using a Hunter Labs Colorflex instrument with D65/10° illumination, with a L* value of about 14.3, a* value of about 10.8, and a b* value of about 2.3. In some embodiments, a composition of the present invention, upon application to skin of a subject, has a color that has no or minimal yellow appearance and/or is translucent on the skin.

According to some embodiments, provided is a method of preparing a composition of the present invention. In some embodiments, a method of preparing a composition of the present invention comprises combining one or more component(s) together and mixing to provide the composition. In some embodiments, a method of the present invention comprises combining a first mixture and a second mixture that are different from each other, thereby providing the composition. In some embodiments, the first mixture comprises a hydrophobic carrier, optionally an oil, and a stiffening agent and/or the second mixture comprises an acid and a hydrophilic active ingredient. In some embodiments, the method comprises mixing and/or homogenizing one or more component(s) together. In some embodiments, a composition of the present invention is homogenized, optionally using methods known to those of skill in the art.

In some embodiments, a method of preparing a composition of the present invention comprises heating a mixture comprising one or more component(s) of the composition to a temperature above the melting point of a component present in the mixture. In some embodiments, the mixture is heated to a temperature above the melting point for each of the one or more component(s) present in the mixture. In some embodiments, a method of preparing a composition of the present invention comprises heating a mixture to a temperature above the melting point of a stiffening agent (e.g., a wax). In some embodiments, a method of preparing a composition of the present invention comprises heating a mixture to a temperature in a range of 35° C., 40° C., 45° C., or 50° C. to about 55° C., 60° C., 65° C., 70° C., 75° C., 80° C., 85° C., 90° C., 95° C., 100° C., or 105° C., optionally wherein the first temperature is heated to a temperature in a range of about 60° C. to about 75° C. After combining and/or heating, a composition of the present invention may be cooled to a temperature of about 50° C. or less, optionally to a temperature in a range of about 30° C. or 35° C. to about 40° C., 45° C., or 50° C.

In some embodiments, a method of the present invention comprises administering a therapeutically effective amount of a composition of the present invention and/or a hydrophilic active ingredient (e.g., a meso-substituted metalloporphyrin) to a subject. As used herein, the term “therapeutically effective amount” refers to an amount of a hydrophilic active ingredient (e.g., a meso-substituted metalloporphyrin) and/or composition of the present invention that elicits a therapeutically useful response in a subject. Those skilled in the art will appreciate that the therapeutic effects need not be complete or curative, as long as some benefit (e.g., treatment and/or prevention) is provided to the subject.

“Treat,” “treating” or “treatment of” (and grammatical variations thereof) as used herein refer to any type of treatment that imparts a benefit to a subject and may mean that the severity of the subject's disease, disorder, and/or condition is reduced, at least partially improved or ameliorated and/or that some alleviation, mitigation or decrease in at least one clinical symptom associated with the disease, disorder, and/or condition (e.g., a dermatological condition) is achieved and/or there is a delay in the progression of the symptom. In some embodiments, the severity of a symptom associated with a disease, disorder, and/or condition (e.g., a dermatological condition) may be reduced in a subject compared to the severity of the symptom in the absence of a method of the present invention.

In some embodiments, a composition of the present invention and/or a hydrophilic active ingredient may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with a disease, disorder, and/or condition (e.g., a dermatological condition) and/or a reduction in the severity of the onset of symptom associated with a disease, disorder, and/or condition (e.g., a dermatological condition) relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention.

In some embodiments, a composition of the present invention and/or a hydrophilic active ingredient (e.g., a meso-substituted metalloporphyrin) may be administered in a prevention effective amount. A “prevention effective” amount as used herein is an amount that is sufficient to prevent (as defined herein) a symptom associated with a disease, disorder, and/or condition (e.g., a dermatological condition) in a subject. Those skilled in the art will appreciate that the level of prevention need not be complete, as long as some benefit is provided to the subject. In some embodiments, a prevention effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention. For example, the subject has findings typically associated with dermatological condition, is suspected to have a dermatological condition, and/or the subject has a dermatological condition.

In some embodiments, a method of treating and/or preventing a disease, disorder, and/or condition (e.g., a dermatological condition) in a subject is provided, the method comprising topically administering to the skin of the subject a composition of the present invention. In some embodiments, the disease, disorder, and/or condition is an inflammatory skin condition. In some embodiments, the disease, disorder, and/or condition is selected from eczema (e.g., atopic dermatitis), psoriasis, pruritus, skin cancer (basal cell), rosacea, acne, and/or arthropod bites or stings.

A method of the present invention may locally deliver a hydrophilic active ingredient (e.g., a meso-substituted metalloporphyrin) to the skin of the subject. In some embodiments, a hydrophilic active ingredient (e.g., a meso-substituted metalloporphyrin) is not systemically absorbed and/or has low distribution to the brain and/or other organs. In some embodiments, no more than about 2% or 1% of the amount of a hydrophilic active ingredient (e.g., a meso-substituted metalloporphyrin) administered to the subject is systemically absorbed. In some embodiments, a hydrophilic active ingredient (e.g., a meso-substituted metalloporphyrin) and/or a metabolite and/or derivative thereof may be systemically absorbed in an amount that is below the level of detection for the hydrophilic active ingredient and/or a metabolite and/or derivative thereof using an assay, such as, but not limited to, a high-performance liquid chromatography (HPLC) assay. In some embodiments, a hydrophilic active ingredient and/or a metabolite and/or derivative thereof may be systemically absorbed in an amount that results in a plasma level that is less than 1 nM of the hydrophilic active ingredient and/or a metabolite and/or derivative thereof, optionally as determined using an assay, such as, but not limited to, a high-performance liquid chromatography (HPLC) assay or undetectable using such an assay.

A method of the present invention may comprise topically applying a composition of the present invention to the skin (e.g., skin affected by a dermatological condition) of a subject 1, 2, 3, 4, 5, 6, 7, or more times a day for a period of time (e.g., 1, 2, 3, 7, 14, 21, 28, or 30 consecutive or non-consecutive day(s)). In some embodiments, a method of the present invention comprises topically applying a composition of the present invention to the skin of a subject 1, 2, 3, 4, 5, 6, 7, or more times a day until the dermatological condition is alleviated and/or as needed and/or desired. In some embodiments, a method of the present invention comprises topically applying a composition of the present invention to the skin of a subject one or two times a day and/or when necessary (e.g., when itching occurs and/or when a lesion is present), optionally up to 3, 4, 5, or 6 times a day. In some embodiments, a thin layer of the composition and/or an amount sufficient to cover a lesion on the skin may be applied onto the skin of the subject.

A composition and/or method of the present invention may reduce the severity of a dermatological condition in a subject by at least about 25% (e.g., about 25%, 30%, 35%, 40%, 45%, 50%, 55%, 60%, 65%, 70%, 75%, 80%, 85%, 90%, 95%, or 100%) in about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The severity of the dermatological condition may be determined in accordance with the EASI (Eczema Area and Severity Index), ADSI (Atopic Dermatology Severity Index), SCORAD (SCORing Atopic Dermatitis), PASI (Psoriasis Area Severity Index), and/or by Investigator's Static Global Assessment Scale (ISGA) of a physician and/or the subject. In some embodiments, a composition and/or method of the present invention may reduce the severity of a dermatological condition in a subject by at least about 25% compared to the severity of a dermatological condition in a subject (e.g., a control subject or the subject receiving the composition) in the absence of a method and/or composition of the present invention and/or compared to a current commercial treatment.

In some embodiments, a composition and/or method of the present invention may reduce inflammation and/or one or more symptoms associated with inflammation in the skin of a subject by at least about 25% (e.g., about 25%, 30%, 35%, 40%, 45%, 50%, 55%, 60%, 65%, 70%, 75%, 80%, 85%, 90%, 95%, or 100%) in about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a composition and/or method of the present invention may reduce inflammation and/or one or more symptoms associated with inflammation in the skin of a subject by at least about 25% compared to the inflammation and/or one or more symptoms associated with inflammation in the skin of a subject in the absence of a method and/or composition of the present invention and/or compared to a current commercial treatment.

In some embodiments, a composition and/or method of the present invention may reduce the number and/or size of lesions present on the subject by at least about 25% (e.g., about 25%, 30%, 35%, 40%, 45%, 50%, 55%, 60%, 65%, 70%, 75%, 80%, 85%, 90%, 95%, or 100%) in about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a composition and/or method of the present invention may reduce the number and/or size of lesions present on the subject by at least about 25% compared to the number and/or size of lesions present on a subject in the absence of a method and/or composition of the present invention and/or compared to a current commercial treatment.

In some embodiments, a composition and/or method of the present invention may improve the esthetic appearance of the skin (e.g., reduce redness, dryness, scaling, and/or blotching) compared to the esthetic appearance of the skin in the absence of a method and/or composition of the present invention and/or compared to a current commercial treatment. In some embodiments, a composition and/or method of the present invention may reduce wrinkles and/or other signs of ageing on the skin of a subject compared to the amount of wrinkles and/or other signs of ageing on the skin of a subject in the absence of a method and/or composition of the present invention and/or compared to a current commercial treatment. In some embodiments, a composition and/or method of the present invention may protect against and/or reduce the effects of UV radiation on the skin of a subject.

In some embodiments, a composition and/or method of the present invention may be useful for treating keloids and/or preventing regrowth of keloids (e.g., following surgical removal).

In some embodiments, a composition and/or method of the present invention may reduce the rate of reoccurrence of the dermatological condition in the subject compared to the rate of recurrence of the same type of dermatological condition in the absence of administering a composition of the present invention. The rate of recurrence may be determined using methods known to those of skill in the art. For example, after treatment of skin affected by the dermatological condition, the number of lesions may be visually determined after a given period of time to determine the rate of recurrence. In some embodiments, a composition and/or method of the present invention may provide a remission period for a specific skin lesion and/or skin condition of about 1, 2, 3, 4, 5, 6, 7, 8, 9, 10, 11, 12, 12, 14, 15, 16, 17, 18, 19, 20, 21, 22, 23, or 24 days, weeks, and/or months.

In some embodiments, a composition of the present invention may further comprise at least one additional therapeutic agent and/or a method of the present invention may further comprise administering at least one additional therapeutic agent. Example additional therapeutic agents include, but are not limited to, a moisturizer, UV protecting agent (e.g., titanium dioxide), anti-itch agent, anti-inflammation agent (e.g., a steroid), calcineurin inhibitor (e.g., pimecrolimus), and/or sodium channel blocker), a topical antibiotic (e.g., clindamcin, retinoids, and/or benzoyl peroxide), and/or an anti-acne agent (e.g., salicylic acid). In some embodiments, a composition of the present invention and an additional therapeutic agent are separately administered to a subject sequentially or concurrently.

A composition and/or method of the present invention may reduce and/or stop itching (e.g., pruritus) of the skin to which the composition is applied within about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 seconds or minutes. In some embodiments, a composition and/or method of the present invention may reduce and/or stop itching (e.g., pruritus) of the skin to which the composition is applied within about 1, 5, 10, 15, or 20 seconds to about 30 or 45 seconds or about 1, 2, 5, 10, 15, 20, 25, or minutes. In some embodiments, the composition and/or method reduces and/or stops itching of the skin to which the composition is applied for at least about 1, 2, 3, 4, 5, 6, 7, 8, 9, 10, 11, 12, 13, 14, 15, 16, 17, 18, 19, 20, 21, 22, 23, or 24 hours and/or days, or optionally permanently. In some embodiments, the composition and/or method may reduce and/or stop itching of the skin to which the composition is applied for about 1, 2, or 4 to about 5, 6, 8, 10, 12, 16, or 24 hours. In some embodiments, a skin lesion (e.g. an atopic dermatitis lesion, cancerous lesion, and/or psoriatic plaque) may be present on the skin and the composition and/or method may be applied to the lesion and may reduce and/or stop itching in less than about 30 seconds or minutes and/or may reduce and/or stop itching of the skin to which the composition is applied for about 1, 2, 3, 4, 5, 6, 7, 8, 9, 10, 11, 12, 13, 14, 15, 16, 17, 18, 19, 20, 21, 22, 23, or 24 hours, or optionally permanently.

The present invention is explained in greater detail in the following non-limiting examples.

### EXAMPLES

### Example 1

Three formulations were tested for in vitro permeation (IVP): a 0.1% BMX-010 gel (see Table 1 for composition), a 0.1% BMX-010 cream (see Table 2 for composition), and a solution of BMX-010 in dimethyl sulfoxide or DMSO (0.1% BMX-010, 99.9% DMSO).

The details for Franz diffusion cell experiments to evaluate BMX-010 delivery to human cadaver skin from various formulations were as follows:
Receptor well volume: 3 mL, stirred with magnetic bar using external drive
Skin area: 0.55 cm²
Receptor fluid: Phosphate buffered saline (PBS) with 0.01% sodium azide
Volume of formulation applied to skin: 10 mcL or ˜18 mg/cm²
Skin surface temperature: Controlled to 32±1.0° C.
Receptor well sampling: 300 mcL at 4, 8, 24, and 48 hours with addition of fresh receptor fluid after each sample.

The test formulations were applied using a blunt glass rod. At the final time point, the skin surface was wiped and washed then tape stripped 20 times to remove any residual formulation and the stratum corneum. The epidermis and dermis layers were then separated; 1 minute on a 60° C. hot plate was used to facilitate separation, if needed.

The BMX-010 was extracted from epidermis and dermis samples. These extracts plus the receptor well samples were analyzed by HPLC.

The median assay values from 6 Franz diffusion cells per formulation, using cadaver skin from a single donor are provided in Table 3.

TABLE 3
Permeation results for a BMX-010 gel, cream or solution.

| | Amount in Epidermis, | Amount in Dermis, |
|---|---|---|
| Formulation | ng/cm² | ng/cm² |
| 0.1% BMX-010 Gel | 34.5 | 37.0 |
| 0.1% BMX-010 Cream | 23.5 | 28.0 |
| 0.1 BMX-010 DMSO | 1042 | 182 |

No BMX-010 was detected in the receptor fluid for the cream or gel (<limit of quantitation of 2 ng/mL). The amounts of BMX-010 that reached the receptor fluid from the DMSO solution were 1.2, 3.4, 8.9, and 10.2 mcg/cm² after 4, 8, 24, and 48 hours, respectively. DMSO significantly increased BMX-010 to the skin. Based on the lack of BMX-010 measured in the receptor phase for the gel and cream, future testing focused on the levels in the epidermis and/or dermis.

### Example 2

Five formulations were tested for in vitro permeation (IVP), which were: a 0.1% BMX-010 cream (see Table 2 for composition) and each of the formulations in Table 4.

TABLE 4
Tested BMX-010 ointment formulations.

| Ingredient | Vehicle 0 | Vehicle B | Vehicle E | Vehicle F |
|---|---|---|---|---|
| Micronized | 0.50 | 0.50 | 0.50 | 0.50 |
| BMX-010 | | | | |
| Oleic acid, | 0.015 | 0.015 | 0.015 | 0.015 |
| NF | | | | |
| Oleyl | 5.00 | 5.00 | — | 0.10 |
| alcohol, | | | | |
| NF | | | | |
| Squalane, NF | 6.00 | — | — | — |
| Mineral oil, | — | 6.00 | 11.00 | 15.00 |
| USP | | | | |
| Paraffin, NF | 2.00 | 5.00 | 5.00 | 5.00 |
| Petrolatum, | q.s. | q.s. | q.s. | q.s. |
| USP | | | | |

q.s. refers to quantity sufficient to total 100% by weight.

The details for the Franz diffusion cell experiments were the same as for Example 1, but the assay was performed using LCMS.

The median assay values from 9 Franz diffusion cells per formulation, using cadaver skin from a single donor, are provided in Table 5.

TABLE 5
Permeation results for a BMX-010 cream and BMX-010 ointments.

| | Amount in Epidermis, | Amount in Dermis, |
|---|---|---|
| Formulation | ng/cm² | ng/cm² |
| 0.03% Cream | 367 | 11.1 |
| 0.5% Vehicle 0 | 87.1 | 25.1 |
| Ointment | | |
| 0.5% Vehicle B | 147 | 41.3 |
| Ointment | | |
| 0.5% Vehicle E | 547 | 101 |
| Ointment | | |

The ointments listed in Table 5 delivered higher amounts to the dermis than the cream formulation.

The median assay values from 9 additional Franz diffusion cells per formulation, using cadaver skin from a single donor, are provided in Table 6.

TABLE 6
Additional permeation results for a BMX-010 cream and BMX-010
ointments.

| | Amount in Epidermis, | Amount in Dermis, |
|---|---|---|
| Formulation | ng/cm² | ng/cm² |
| 0.03% Cream | 482 | 34.5 |
| 0.5% Vehicle E | 1360 | 155 |
| Ointment | | |
| 0.5% Vehicle F | 340 | 40 |
| Ointment | | |

Ointment Vehicle E delivered higher amounts to the dermis than the cream formulation or Ointment Vehicle F. The ointments listed in Table 6 delivered higher amounts to the dermis than the cream formulation.

### Example 3

Three formulations were tested for in vitro permeation (IVP), which are those provided in Table 7.

TABLE 7
Formulations using Vehicle E with varying amounts of BMX-010.

| Ingredient | 0.03% | 0.1% | 0.5% |
|---|---|---|---|
| Micronized | 0.03 | 0.10 | 0.50 |
| BMX-010 | | | |
| Oleic acid, | 0.015 | 0.015 | 0.015 |
| NF | | | |
| Mineral oil, | 11.0 | 11.0 | 11.00 |
| USP | | | |
| Paraffin, NF | 5.00 | 5.00 | 5.00 |
| Petrolatum, | q.s. | q.s. | q.s. |
| USP | | | |

The details for the Franz diffusion cell experiments to evaluate BMX-010 delivery to human cadaver skin from the formulations were the same as for Example 1, but the assay was performed using LCMS.

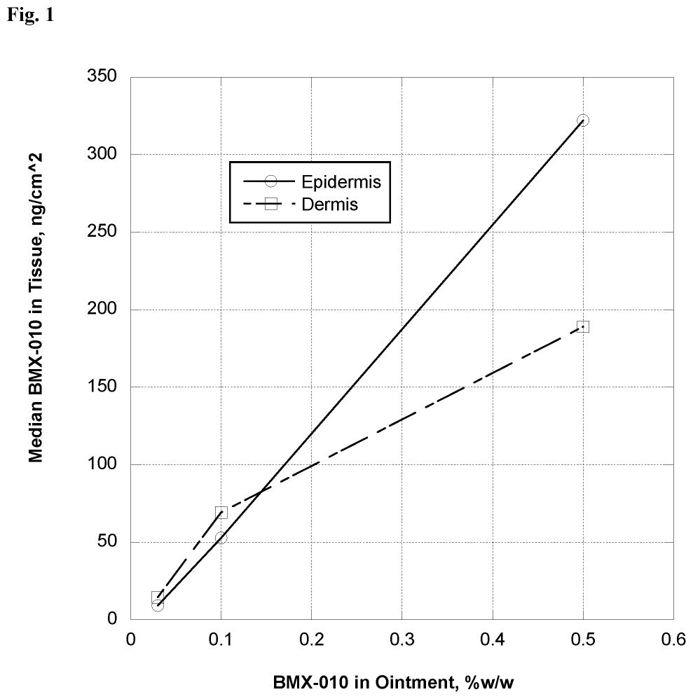

The median assay values from 9 Franz diffusion cells per formulation, using cadaver skin from a single donor, are summarized in FIG. 1. As can be seen in FIG. 1, as the BMX-010 concentration increased in the formulation, the amounts delivered to the epidermis and dermis also increased. These data demonstrate that the ointment formulations, with suspended BMX-010 particles, can deliver the drug substance to the skin over a wide range of drug concentrations.

### Example 4

The stability of BMX-010 alone or in a composition was tested. Compositions with three different amounts of BMX-010 were tested. The compositions included BMX-010 in an amount of 0.03%, 0.1%, or 0.5% by weight of the composition (wt %) along with 0.015 wt % oleic acid, NF; 11 wt % mineral oil, USP; 5 wt % paraffin, NF; and petrolatum, USP in an amount sufficient to total 100 wt %. Each composition was stored in a 15 gram laminate tube.

### Sample Preparation for Testing Ointment Stability:

1. Add target amount of ointment to a 50 mL plastic centrifuge tube (see Table 8).
2. Add 3.5-4 mL hexane, wrap parafilm around tube threads and cap over the parafilm.
3. Vortex tube until dissolved and no ointment globules are visible.
4. Remove cap and parafilm, add specified amount of water (gravimetrically, Table 8), apply fresh parafilm and cap.
5. Place tube on shaker/sample rotator and mix for 45 minutes at a suitable speed.
6. Place tube upright in a holder and examine the ointment layer at the top to ensure it is white to off-white with no significant purple/brown color.
7. Withdraw a sample from the bottom (dark brown/purple) aqueous layer and vial.

TABLE 8
Sample preparation conditions.

| BMX-010 Content, % w/w | Target Ointment Weight (g) | Water (mL) |
|---|---|---|
| 0.03% | 0.75 | 10 |
| 0.10% | 0.25 | 10 |
| 0.50% | 0.175 | 35 |

### Chromatographic System Parameters:

Waters Acquity UPLC (or equivalent)

| Column | Acquity ® HSS T3, | | | |
|---|---|---|---|---|
| | 2.1 × 150 mm, 1.8 μm | | | |
| | P/N: 0186005221 | | | |
| Column Temperature | 60° C. ± 5° C. | | | |
| Sample Temperature | 20° C. ± 5° C. | | | |
| Needle Depth | 6.0 mm | | | |
| Sample Manager 1: | 94% Water:6% MeOH:0.1% TFA | | | |
| Sample Manager 2: | Methanol | | | |
| Pump Seal Wash | 94% Water:6% MeOH:0.1% TFA | | | |
| Mobile Phase A | 0.1% TFA in Water | | | |
| Mobile Phase B | 0.1% TFA in Methanol | | | |
| Flow Rate | 0.2 mL/Minute | | | |
| | Time | % A | % B | Curve |
| Pump settings¹ | initial | 94² | 6² | initial |
| | 10.0 | 94² | 6² | 6 |
| | 15.0 | 85 | 15 | 6 |
| | 30.0 | 60 | 40 | 6 |
| | 35.0 | 0 | 1000 | 6 |
| | 37.0 | 0 | 100 | 6 |
| | 37.1 | 94² | 6² | 6 |

| | |
|---|---|
| Injection volume | 6.0 μL |
| Detector | 2D Data channel 454 nm, 1.2 nm bandwidth |
| Next Inject Delay | 10 minutes |
| Run Time | 37 minutes |
| Sampling Rate | 2 points/second |
| Quantitation | Peak area |
| Integration | 4 to 37 minutes |

¹Enable “Gradient Start 275 μL before injection”-in IM-QSM/Misc
²May be altered to adjust retention time/resolution

Viscosity was measured using a Brookfield HA DV-3T using the following parameters: Spindle: CPA-52Z, Test Speed: 5 RPM, Sample Size: ˜0.5 g, Temperature: 25° C.±0.1° C., Equilibration: 5 minutes, and Read Time: 2 minutes.

The stability results are provided in Tables 9-11.

TABLE 9
Stability results for the 0.5% BMX-010 Ointment.

| Storage | | Results, 1 | Results, 3 | Results, 6 |
|---|---|---|---|---|
| Condition | Results | Month | Months | Months |
| Initial | | | | |
| Appearance | Pass | | | |
| Assay, % LC | 99.7 | | | |
| Impurities, | 0.55 | | | |
| Area % | | | | |
| Viscosity, cP | 26,450 | | | |
| D50, μm | NT | | | |
| 25° C./60% | | | | |
| Appearance | | Pass | Pass | Pass |
| Assay, % LC | | 101.2 | 100.3 | 102.5 |
| Impurities, | | 0.77 | 0.61 | 0.61 |
| Area % | | | | |
| Viscosity, cP | | 32,580 | 33,500 | 29,210 |
| D50, μm | | 1.11 | 1.13 | 1.11 |
| 30° C./65% | | | | |
| Appearance | | Pass | Pass | Pass |
| Assay, % LC | | 100.7 | 100.8 | 102.0 |
| Impurities, | | 0.71 | 0.62 | 0.64 |
| Area % | | | | |
| Viscosity, cP | | 32,230 | 31,430 | 31,270 |
| D50, μm | | 1.11 | 1.14 | 1.11 |

TABLE 10
Stability results for the 0.1% BMX-010 Ointment.

| Storage | | Results, 1 | Results, 3 | Results, 6 |
|---|---|---|---|---|
| Condition | Results | Month | Months | Months |
| Initial | | | | |
| Appearance | Pass | | | |
| Assay, % LC | 99.5 | | | |
| Impurities, | 0.61 | | | |
| Area % | | | | |
| Viscosity, cP | 30,040 | | | |
| D50, μm | 1.23 | | | |
| 25° C./60% | | | | |
| Appearance | | Pass | Pass | |
| Assay, % LC | | 98.4 | 102.9 | |
| Impurities, | | 0.63 | 0.60 | |
| Area % | | | | |
| Viscosity, cP | | 26,790 | 30,560 | |
| D50, μm | | 1.48 | 1.42 | |
| 30° C./65% | | | | |
| Appearance | | Pass | Pass | |
| Assay, % LC | | 98.9 | 102.6 | |
| Impurities, | | 0.65 | 0.62 | |
| Area % | | | | |
| Viscosity, cP | | 29,290 | 26,110 | |
| D50, μm | | 1.43 | 1.39 | |

TABLE 11
Stability results for the 0.03% BMX-010 Ointment.

| Storage | | Results, 1 | Results, 3 | Results, 6 |
|---|---|---|---|---|
| Condition | Results | Month | Months | Months |
| Initial | | | | |
| Appearance | Pass | | | |
| Assay, % LC | 99.1 | | | |
| Impurities, | 0.65 | | | |
| Area % | | | | |
| Viscosity, cP | 26,230 | | | |
| D50, μm | 2.12 | | | |
| 25° C./60% | | | | |
| Appearance | | Pass | Pass | |
| Assay, % LC | | 99.4 | 97.3 | |
| Impurities, | | 0.62 | 0.65 | |
| Area % | | | | |
| Viscosity, cP | | 27,580 | 27,030 | |
| D50, μm | | 2.17 | 2.05 | |
| 30° C./65% | | | | |
| Appearance | | Pass | Pass | |
| Assay, % LC | | 99.4 | 98.2 | |
| Impurities, | | 0.63 | 0.67 | |
| Area % | | | | |
| Viscosity, cP | | 29,920 | 28,060 | |
| D50, μm | | 3.26 | 1.83 | |

BMX-010 alone was packaged in an amber glass vial that was layered in nitrogen and closed with a Teflon® cap. The vial was placed in a first 3 mL low density polyethylene (LDPE) bag with a 1/6 desiccant and the bag was twist tie closed. The first bag was placed in a second 3 mL LDPE bag, twist tie closed, and placed in a high density polyethylene (HDPE) container. The stability results for BMX-010 alone are provided in Table 12.

TABLE 12
Stability results for BMX-010 alone.

| Test | | | | | |
|---|---|---|---|---|---|
| (Test | | Storage | Interval | | |
| Method) | Specifications | Condition | T = 0 | 3 Month | 6 Month |
| Appearance | Report Result | −20° C. | Brown Solid | Brown Solid | Brown Solid |
| (TM.795) | | 25° C./60% RH | | Brown Solid | Brown Solid |
| | | 40° C./75% RH | | Brown Solid | Brown Solid |
| Assay by | Report Result | −20° C. | 97.5% | 101.4% | 100.7% |
| UPLC | (Wt % of an | 25° C./60% RH | | 100.3% | 99.9% |
| (TM.04347) | anhydrous | 40° C./75% RH | | 100.2% | 97.9%** |
| | basis, sum of | | | | |
| | Atropisomers) | | | | |
| Total | ≤5.0% | −20° C. | 0.67% | 0.63% | 0.63% |
| Impurities | (Area %) | 25° C./60% RH | | 0.81% | 0.88% |
| by HPLC | | 40° C./75% RH | | 1.3% | 1.6%** |
| (TM.04347) | | | | | |
| Water | Report Result | −20° C. | 6.9% | 7.0% | 7.4% |
| Content by | | 25° C./60% RH | | 7.3% | 7.7% |
| Karl | | 40° C./75% RH | | 7.5% | 8.5% |
| Fischer | | | | | |
| (TM.04998) | | | | | |
| XRPD | Report Result | −20° C. | Amorphous | Semi- | Amorphous |
| (TM.60) | | | | Crystalline | |
| | | 25° C./60% RH | | Semi- | Amorphous |
| | | | | Crystalline | |
| | | 40° C./75% RH | | Semi- | Amorphous |
| | | | | Crystalline | |
| PSD | Report Result | −20° C. | D₁₀: 0.61 μm | D₁₀: 0.59 μm | D₁₀: 0.59 μm* |
| (TM.05869) | | | D₅₀: 1.2 μm | D₅₀: 1.1 μm | D₅₀: 1.1 μm* |
| | | | D₉₀: 2.0 μm | D₉₀: 2.0 μm | D₉₀: 1.9 μm* |
| | | 25° C./60% RH | | D₁₀: 0.60 μm | D₁₀: 0.62 μm* |
| | | | | D₅₀: 1.1 μm | D₅₀: 1.2 μm* |
| | | | | D₉₀: 2.0 μm | D₉₀: 2.0 μm* |
| | | 40° C./75% RH | | D₁₀: 0.60 μm | D₁₀: 0.61 μm* |
| | | | | D₅₀: 1.2 μm | D₅₀: 1.2 μm* |
| | | | | D₉₀: 2.1 μm | D₉₀: 2.1 μm* |

*= See DEV-23472
**= See DEV-23401

### Example 5

Exemplary formulation including BMX-010 and semipolar oils and waxes are provided in Table 13.

TABLE 13
Exemplary formulations.

| | Example 1, | Example 2, | Example 3, |
|---|---|---|---|
| Ingredient | % w/w | % w/w | % w/w |
| Micronized BMX- | 0.03-0.50 | 0.03-0.50 | 0.03-0.50 |
| 010 | | | |
| Oleic acid, NF | 0.015 | 0 | 0.01 |
| Undecylenic acid, | 0 | 0.025 | 0.01 |
| USP | | | |
| Medium-chain | 20 | 20 | 20 |
| triglycerides, | | | |
| NF | | | |
| Octyldodecanol, | 12 | 12 | 0 |
| NF | | | |
| Isopropyl | 0 | 10 | 0 |
| myristate, NF | | | |
| Olive oil, NF | 20 | 0 | 15 |
| White wax, NF | 10 | 10 | 10 |
| (Beeswax) | | | |
| Cetyl esters wax, | 10 | 10 | 10 |
| NF | | | |
| Glyceryl | 5 | 2.5 | 5 |
| monostearate, | | | |
| NF | | | |
| Hydrogenated | 2 | 1.5 | 2 |
| Castor Oil, NF | | | |
| Coconut Oil, NF | q.s. to 100% | q.s. to 100% | q.s. to 100% |

The foregoing is illustrative of the present invention, and is not to be construed as limiting thereof. The invention is defined by the following claims, with equivalents of the claims to be included therein.

1. A composition comprising:
a hydrophilic active ingredient in an amount of about 0.005% to about 10% by weight of the composition;
a hydrophobic carrier in an amount of about 60% to about 99% by weight of the composition;
and an acid in an amount of about 0.001% to about 2.5% by weight of the composition.

2. The composition of claim 1, wherein the hydrophilic active ingredient is a meso-substituted metalloporphyrin.

3. The composition of claim 1, wherein the hydrophilic active ingredient has a structure represented by Formula I: 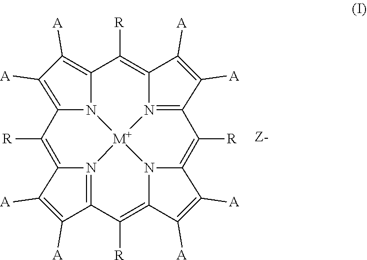
wherein:
each R is independently substituted or unsubstituted aryl, heteroaryl, cycloalkyl, or heterocycloalkyl;
each A is an independently selected hydrogen, or an electron-withdrawing or electron donating group;
M⁺ is a metal or is absent in which case a hydrogen is added to each of the two nitrogens required to correct valency; and
Z⁻ is a counterion.

4. The composition of claim 1, wherein the hydrophilic active ingredient is MnTE-2-PyP⁵⁺ and has the structure: 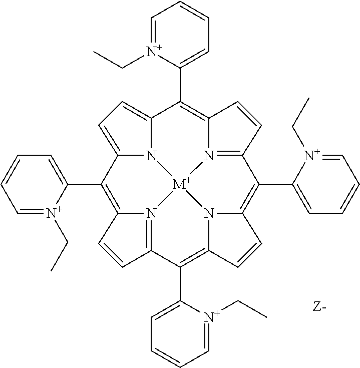
wherein Z⁻ is a counterion and M⁺ is manganese.

5. The composition of claim 1, wherein the hydrophilic active ingredient has a structure of Formula A1 or A2: 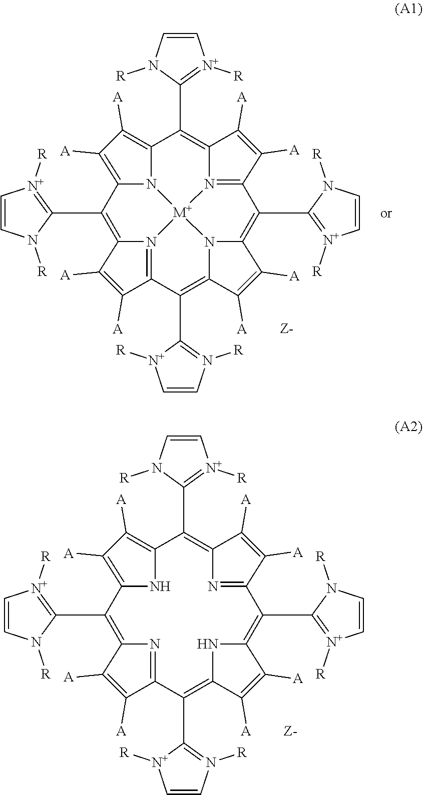
wherein:
each R is, independently, a C₁₋₁₂ alkyl;
each A is, independently, a hydrogen or an electron withdrawing group;
M⁺ is a metal selected from the group consisting of manganese, iron, copper, cobalt, nickel and zinc; and
Z⁻ is a counterion.

6. The composition of claim 1, wherein the hydrophilic active ingredient is BMX-010 and has the structure:

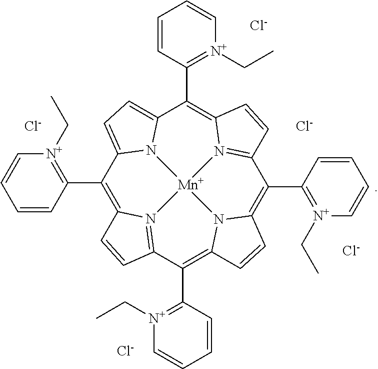

7. The composition of claim 1, wherein the hydrophilic active ingredient is micronized and/or wherein the hydrophilic active ingredient has a D90 particle size of less than 50 microns.

8. The composition of claim 1, further comprising a stiffening agent and/or an oil.

9. The composition of claim 1, wherein the hydrophobic carrier and/or acid has a Hildebrand solubility parameter (δ) of less than 26 MPa^{1/2}.

10. The composition of claim 1, wherein the acid has a Hydrophile Lipophile Balance of 10 or less.

11. (canceled)

12. The composition of claim 1, wherein the acid is present in the composition in an amount of less than about 0.15% by weight of the composition.

13. The composition of claim 1, further comprising stiffening agent that has a melting point in a range of about 35° C. to about 105° C. and/or is microcrystalline.

14. The composition of claim 8, wherein the stiffening agent is a paraffin wax and/or the oil is mineral oil.

15. The composition of claim 1, further comprising:
an oil in an amount of about 5% to about 25% by weight of the composition; and
a stiffening agent in the composition in an amount of about 1% to about 10% by weight of the composition.

16. The composition of claim 15, wherein the hydrophobic carrier is a petrolatum, the stiffening agent is a paraffin wax, the oil is mineral oil, and the acid is oleic acid.

17. The composition of claim 1, wherein the composition is devoid of squalene, cyclomethicone, and/or oleyl alcohol.

18. The composition of claim 1, wherein the composition is an ointment.

19. The composition of claim 1, wherein the composition does not separate after storage for about 1 month at about with about 65% relative humidity.

20.-22. (canceled)

23. A method of preparing a composition, the method comprising:
combining a hydrophobic carrier, an acid, and a hydrophilic active ingredient, thereby providing the composition.

24.-26. (canceled)

27. A method of treating and/or preventing a disease, disorder, and/or condition in a subject, the method comprising topically administering to the skin of the subject the composition of claim 1.

28.-35. (canceled)
